(19) United States
(12) Patent Application Publication
(10) Pub. No.: US 2009/0215779 A1
(43) Pub. Date: Aug. 27, 2009

(54) OXADIAZOLE DERIVATIVES AS DGAT INHIBITORS

(76) Inventors: Roger John Butlin, Cheshire (GB); Clive Philip Green, Cheshire (GB); William McCoull, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 11/917,027

(22) PCT Filed: Jun. 6, 2006
(86) PCT No.: PCT/GB2006/002067

§ 371 Date: Aug. 18, 2008

(30) Foreign Application Priority Data
Jun. 11, 2005 (GB) 0511851.8
Sep. 16, 2005 (GB) 0518924.6

(51) Int. Cl. A61K 31/4245; C07D 413/12; C07D 413/14; C07D 271/10; A61K 31/5377; A61K 31/496; A61P 31/10

(52) U.S. Cl. 514/236.2; 544/138; 544/131; 548/143; 544/367; 514/364; 514/254.03

(57) ABSTRACT

Compounds of Formula (I):

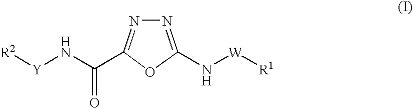

wherein R¹-R², W and Y are as described in the specification,
and their salts and pro-drugs, are inhibitors of DGAT and are thereby useful in the treatment of, for example, obesity. Processes for preparing compounds of formula (I) are also described.

The present invention relates to compounds which inhibit acetyl CoA(acetyl coenzyme A):diacylglycerol acyltransferase (DGAT1) activity, processes for their preparation, pharmaceutical compositions containing them as the active ingredient, methods for the treatment of disease states associated with DGAT1 activity, to their use as medicaments and to their use in the manufacture of medicaments for use in the inhibition of DGAT1 in warm-blooded animals such as humans. In particular this invention relates to compounds useful for the treatment of type II diabetes, insulin resistance, impaired glucose tolerance and obesity in warm-blooded animals such as humans, more particularly to the use of these compounds in the manufacture of medicaments for use in the treatment of type II diabetes, insulin resistance, impaired glucose tolerance and obesity in warm-blooded animals such as humans.

Acyl CoA:diacylglycerol acyltransferase (DGAT) is found in the microsomal fraction of cells. It catalyzes the final reaction in the glycerol phosphate pathway, considered to be the main pathway of triglyceride synthesis in cells by facilitating the acylation of a diacylglycerol with a fatty acyl CoA, resulting in the formation of triglyceride. Although it is unclear whether DGAT is rate-limiting for triglyceride synthesis, it catalyzes the only step in the pathway that is committed to producing this type of molecule [Lehner & Kuksis (1996) Biosynthesis of triacylglycerols. Prog. Lipid Res. 35: 169-201].

Two DGAT genes have been cloned and characterised. Both of the encoded proteins catalyse the same reaction although they share no sequence homology. The DGAT1 gene was identified from sequence database searches because of its similarity to acyl CoA:cholesterol acyltransferase (ACAT) genes. [Cases et al (1998) Identification of a gene encoding an acyl CoA:diacylglycerol acyltransferase, a key enzyme in triacylglycerol synthesis. Proc. Natl. Acad. Sci. USA 95: 13018-13023]. DGAT1 activity has been found in many mammalian tissues, including adipocytes.

Because of the previous lack of molecular probes, little is known about the regulation of DGAT1. DGAT1 is known to be significantly up-regulated during adipocyte differentiation.

Studies in gene knockout mice has indicated that modulators of the activity of DGAT1 would be of value in the treatment of type II diabetes and obesity. DGAT1 knockout (Dgat1^{-/-}) mice, are viable and capable of synthesizing triglycerides, as evidenced by normal fasting serum triglyceride levels and normal adipose tissue composition. Dgat1^{-/-} mice have less adipose tissue than wild-type mice at baseline and are resistant to diet-induced obesity. Metabolic rate is ˜20% higher in Dgat1^{-/-} mice than in wild-type mice on both regular and high-fat diets [Smith et al (2000) Obesity resistance and multiple mechanisms of triglyceride synthesis in mice lacking DGAT. Nature Genetics 25: 87-90]. Increased physical activity in Dgat1^{-/-} mice partially accounts for their increased energy expenditure. The Dgat1^{-/-} mice also exhibit increased insulin sensitivity and a 20% increase in glucose disposal rate. Leptin levels are 50% decreased in the Dgat1^{-/-} mice in line with the 50% decrease in fat mass.

When Dgat1^{-/-} mice are crossed with ob/ob mice, these mice exhibit the ob/ob phenotype [Chen et al (2002) Increased insulin and leptin sensitivity in mice lacking acyl CoA:diacylglycerol acyltransferase J. Clin. Invest. 109:1049-1055] indicating that the Dgat1^{-/-} phenotype requires an intact leptin pathway. When Dgat1^{-/-} mice are crossed with Agouti mice a decrease in body weight is seen with normal glucose levels and 70% reduced insulin levels compared to wild type, agouti or ob/ob/Dgat1^{-/-} mice.

Transplantation of adipose tissue from Dgat1^{-/-} mice to wild type mice confers resistance to diet-induced obesity and improved glucose metabolism in these mice [Chen et al (2003) Obesity resistance and enhanced glucose metabolism in mice transplanted with white adipose tissue lacking acyl CoA:diacylglycerol acyltransferase J. Clin. Invest. 111: 1715-1722].

International Patent Application WO2004/047755 (Tularik and Japan Tabacco) describes fused bicyclic nitrogen-containing heterocycles which are inhibitors of DGAT-1. JP2004-67635 (Otsuka Pharmaceuticals) describes thiazoleamido substituted phenyl compounds which are further substituted with alkylphosphonates and which inhibit DGAT-1. WO2004/100881 (Bayer) describes biphenylamino compounds substituted with imidazole, oxazole or thiazole which inhibit DGAT-1.

Accordingly, the present invention provides a compound of formula (I)

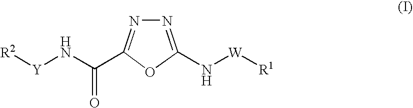

or a salt or prodrug thereof, wherein

R¹ is an optionally substituted aryl or optionally substituted heteroaryl group, wherein the optional substituents are one or more groups selected from a group -Z^{a}, a group —X²—(CR³R⁴)_{q}-Z^{a}, a group —X²—(CR³R⁴)ₐ—X³-Z^{a}, a group —(CR³R⁴)ₐ—X³-Z^{a} and a group R^{f};

W is selected from —C(O)—, —C(O)O—, —C(O)NH— and —C(O)(CR^{A}R^{B})ₖ—;

k is 0 to 4;

R^{A} and R^{B} are independently selected from hydrogen and (1-4C)alkyl, and/or two groups R^{A} and/or R^{B} are joined together to form a (3-8C)cycloalklyl ring;

Y is a direct bond, or a group (CR⁵R⁶), or —X⁶(CR⁵R⁶)ₜ— where each R⁵ and R⁶ is independently selected from hydrogen, (1-4C)alkyl, hydroxy, halo, halo(1-4C)alkyl, amino, (1-4C)alkoxy, cyano(1-4C)alkoxy, (1-4C)haloalkoxy or (1-4C)alkylCONH—, s is an integer of from 1 to 6 and t is an integer of from 1 to 6, provided that the X⁶ atom of the group

—X⁶(CR⁵R⁶)ₜ— is attached to the R² group and that a single sp3 hybridised carbon atom does not carry two or more bonds to a heteroatom unless the heteroatom is a halo;

R² is an optionally substituted aryl, an optionally substituted (3-8C)cycloalkyl, optionally substituted (5-12C)bicycloalkyl, optionally substituted (6-12C)tricycloalkyl or an optionally substituted heterocyclic group, wherein optional substitutents are one or more groups selected from a group -Z, a group —X—(CR⁷R⁸)ᵤ-Z, a group —X—(CR⁷R⁸)ᵥ—X¹-Z or a group —(CR⁷R⁸)ᵥX¹-Z and a group R^{f};

Z and Z^{a} are independently selected from a hydrocarbyl group or a heterocyclic group or a combination thereof, wherein the group Z and Z^{a} is optionally substituted on any available atom by one or more groups selected from R^{f}, or by a group —X⁷—(CR⁹R¹⁰)_{b}R¹¹; X, X¹, X², X³, X⁶ and X⁷ are linking groups independently selected from —C(O)_{X}—, —O—, —S(O)_{y}—, —NR¹²—, —C(O)NR¹²—, —OC(O)NR¹²—, —CH═NO—, —NR¹²C(O)ₓ—, —NR¹²CONR³—, —S(O)₂NR¹²— and —NR¹²S(O)₂— where x is an integer of 1 or 2, y is 0, 1 or 2, and R¹² and R¹³ are independently selected from hydrogen or (1-6C)alkyl;

u and q are independently selected from 0 or an integer of from 1 to 6;

v, a and b are independently selected from an integer of from 1 to 6;

each R³, R⁴, R⁷, R⁸, R⁹ and R¹⁰ is independently selected from hydrogen, (1-4C)allyl, hydroxy, halo, halo(1-4C)alkyl, amino, cyano(1-4C)alkoxy, (1-4C)haloalkoxy, (1-3C)alkylCONH—, carboxy and a carboxylic acid mimic or bioisostere thereof;

R^{f} and R¹¹ are independently at each occurrence selected from halo, haloC₁₋₆alkyl, cyano, nitro, C(O)ₙR¹⁴, a carboxylic acid mimic or bioisostere thereof, OR¹⁴, S(O)ₘR¹⁴, OS(O)₂R⁴, NR¹⁵R¹⁶, C(O)NR¹⁵R¹⁶, OC(O)NR¹⁵R¹⁶, —CH═NOR¹⁴, —NR¹⁵C(O)ₙR¹⁴, —NR¹⁴CONR¹⁵R¹⁶, —N═CR¹⁵R¹⁶, S(O)₂NR¹⁵R¹⁶ and —NR¹⁵S(O)₂R¹⁶ where R¹⁴, R¹⁵ and R¹⁶ are independently selected from hydrogen or optionally substituted hydrocarbyl or optionally substituted heterocyclyl, or R¹⁵ and R¹⁶ together with the nitrogen atom to which they are attached form an optionally substituted ring having from 3 to 10 atoms, which optionally contains further heteroatoms such as S(O)ₘ, oxygen and nitrogen;

n is an integer of 1 or 2,

m is 0 or an integer of 1 or 2.

Suitable optional substituents for hydrocarbyl groups or heterocyclic groups R¹⁴, R¹⁵ and R¹⁶ include halo, halo(1-4C)alkyl (such as trifluoromethyl, difluoromethyl or fluoromethyl), mercapto, hydroxy, (1-6C)alkoxy, oxo, heteroaryloxy, alkenyloxy, alkynyloxy, alkoxyalkoxy (such as (1-4C)alkoxy(2-4C)alkoxy), aryloxy (where the aryl group may be substituted by halo, cyano, nitro, hydroxy(1-4C)alkyl, halo(1-4C)alkyl, amino, (1-4C)alkoxy, (1-4C)haloalkoxy, (1-3C)alkylCONH—, carboxy or a carboxylic acid mimic or bioisostere thereof), cyano, nitro, amino, mono- or di-alkylamino (such as mono- or di(1-4C)alkylamino), alkylamido (such as (1-4C)alkylaminocarbonyl), oximino (for example hydroxyimino or alkyloxyimino), carbamoyl, carboxy or a carboxylic acid mimic or bioisostere thereof, or S(O)ₘR¹⁷ where m is as defined above and R¹⁷ is alkyl (optionally substituted by one or more groups selected from hydroxy, halo, amino, cyano, (1-3C)alkylCONH—, carboxy or a carboxylic acid mimic or bioisostere thereof), (1-6C)alkoxy, (1-6C)alkoxycarbonyl, carbamoyl, (1-6C)alkylaminocarbonyl, halo(1-6C)alkyl (such as trifluoromethyl), (1-6C)alkylsulphonyl, and (1-6C)alkylsulphinyl. Heterocyclic groups R¹⁴, R¹⁵ and R¹⁶ may also be optionally substituted by one or more hydrocarbyl groups such as (1-4C)alkyl.

In this specification the term “alkyl” includes both straight and branched chain alkyl groups but references to individual alkyl groups such as “propyl” are specific for the straight chain version only. An analogous convention applies to other generic terms. Unless otherwise stated the term “alkyl” advantageously refers to chains with 1-10 carbon atoms, suitably from 1-6 carbon atoms, preferably 1-4 carbon atoms.

In this specification the term “alkoxy” means an allyl group as defined hereinbefore linked to an oxygen atom.

It is to be understood that optional substituents on any group may be attached to any available atom as appropriate unless otherwise specified, including heteroatoms provided that they are not thereby quaternised.

In this specification the term “heteroatom” refers to non-carbon atoms such as oxygen, nitrogen or sulphur atoms. In addition, where the heteroatom may have a single valency, it may comprise a halo. The terms “alkenyl” and “alkynyl” refer to unsaturated straight or branched structures, which unless specified otherwise, contain for example from 2 to 10, preferably from 2 to 6 carbon atoms. Cyclic moieties such as cycloalkyl and cycloalkenyl are similar in nature but have at least 3 carbon atoms.

Examples of (1-4C)alkyl include methyl, ethyl, propyl and isopropyl. Examples of (1-6C)alkyl include methyl, ethyl, propyl, isopropyl, t-butyl, pentyl, iso-pentyl, 1-2-dimethylpropyl and hexyl; examples of (2-6C)alkenyl include ethenyl, propenyl, isopropenyl, 2-pentenyl, 3-pentenyl, 4-pentenyl, 2-methylpropenyl and hexenyl; examples of alkenyloxy include ethenyloxy, propenyloxy, isopropenyloxy, 2-pentenyloxy, 3-pentenyloxy, 4-pentenyloxy, 2-methylpropenyloxy and hexenyloxy; examples of (2-6C)alkynyl include ethynyl, propynyl, 2-pentynyl, 3-pentynyl, 4-pentynyl and hexynyl; examples of alkynyloxy include ethynyloxy, propynyloxy, 2-pentynyloxy, 3-pentynyloxy, 4-pentynyloxy and hexynyloxy; examples of (1-4C)alkoxy include methoxy, ethoxy, propoxy, isopropoxy, and tert-butoxy; examples of (1-6C)alkoxy include methoxy, ethoxy, propoxy, isopropoxy, tert-butoxy and pentoxy; examples of alkoxyalkoxy include (1-4C)alkoxy(2-4C)alkoxy such as methoxyethoxy and ethoxyethoxy; examples of (3-8C)cycloalkyl include (3-6C)cycloalkyl (cyclopropyl, cyclobutyl, cyclopentyl, cyclohexyl), cycloheptyl and cycloctyl; examples of (5-12C)bicycloalkyl include norbornyl, bicyclo[2,2,2]octane, decalinyl (bicyclo[4,4,0]decyl (cis and trans), bicyclo[5,3,0]decyl and hydrindanyl (bicyclo[4,3,0]nonyl); examples of (6-12)tricycloalkyl include adamantyl (tricyclo[3,3,1,1]decyl), homoadamantyl (tricyclo[4,3,1,1]undecyl) and isomers of perhydrophenanthrene; examples of halo are chloro, bromo and fluoro; examples of halo(1-6C)alkyl include halo(1-4C)alkyl (such as chloromethyl, fluoroethyl, fluoromethyl, fluoropropyl, fluorobutyl, dichloromethyl, difluoromethyl, 1,2-difluoroethyl and 1,1-difluoroethyl) as well as perhalo(1-6C)alkyl and perhalo(1-4C)allyl (such as trifluoromethyl, pentafluoroethyl, and heptafluoropropyl); examples of halo(1-6C)alkoxy include halo(1-4C)alkoxy (such as chloromethoxy, fluoroethoxy and fluoromethoxy, difluoromethoxy), as well as perhaloalkoxy such as pentafluoroethoxy, trifluoromethoxy and heptafluoropropoxy; examples of hydroxy(1-6C)alkyl include hydroxy(1-4C)alkyl such as hydroxy methyl, 1-hydroxyethyl, 2-hydroxyethyl and 3-hydroxybutyl; examples of cyano(1-4C)alkoxy include cyanomethoxy, 1-cyanoethoxy, 2-cyanoethoxy and 3-cyanobutoxy; example of carboxy(1-6C)alkyl include carboxy(1-4C)alkyl, such as carboxymethyl, carboxyethyl, carboxypropyl and carboxybutyl; examples of (1-6C)alkylcarbonyl include (1-4C)alkylcarbonyl such as methylcarbonyl, ethylcarbonyl, propylcarbonyl, iso-propylcarbonyl and tert-butylcarbonyl; examples of (1-6C)alkylcarbonyloxy include (1-4C)alkylcarbonyloxy such as methylcarbonyloxy, ethylcarbonyloxy, propylcarbonyloxy, iso-propylcarbonyloxy and tert-butylcarbonyloxy; examples of (1-6C)alkoxycarbonyl include (1-4C)alkoxycarbonyl such as methoxycarbonyl, ethoxycarbonyl, propoxycarbonyl, iso-propoxycarbonyl and tert-butoxycarbonyl; examples of (1-6C)alkylthio include methylthio, ethylthio, propylthio, isopropylthio and butylthio; examples of (1-6C)alkylsulfinyl include methylsulfinyl, ethylsulfinyl, propylsulfinyl, isopropylsulfinyl and butylsulfinyl; examples of (1-6C)alkylsulfonyl include methylsulfonyl, ethylsulfonyl, propylsulfonyl, isopropylsulfonyl and butylsulfonyl; examples of (1-6C)alkoxysulfonyl include methoxysulfonyl, ethoxysulfonyl, propoxysulfonyl, isopropoxysulfonyl and butoxysulfonyl; examples of (1-6C)alkylcarbonylamino include (1-4C)alkylcarbonylamino [(1-4C)alkylCONH—] such as tert-butylcarbonylamino and (1-3C)alkylCONH— such as methylcarbonylamino, ethylcarbonylamino, propylcarbonylamino and iso-propylcarbonylamino; examples of (1-6C)alkylaminocarbonyl include (1-4C)alkylaminocarbonyl such as methylaminocarbonyl, ethylaminocarbonyl, propylaminocarbonyl, iso-propylaminocarbonyl and tert-butylaminocarbonyl; examples of di(1-6C)alkylaminocarbonyl include di(1-4C)alkylaminocarbonyl such as dimethylaminocarbonyl, N-methyl-N-ethylaminocarbonyl, diethylaminocarbonyl, N-methyl-N-propylaminocarbonyl and di-isopropylaminocarbonyl; examples of mono-alkyl amino include (1-4C)alkylamino such as methylamino, ethylamino, propylamino, isopropylamino and tert-butylamino; examples of di-alkylamino include di(1-4C)alkylamino, such as dimethylamino, diethylamino, N-methyl-N-ethylamino, N-methyl-N-propylamino and di-isopropylamino;

References to aryl groups include aromatic carbocylic groups such as phenyl and naphthyl, as well as partially aromatic groups such as indenyl and indanyl.

The term “heterocyclyl” or “heterocyclic” includes saturated or unsaturated rings, which may be aromatic, non-aromatic rings or partially aromatic, for example containing from 3 to 20, suitably from 4 to 10 ring atoms, at least one of which is a heteroatom such as oxygen, sulphur or nitrogen. They may be mono- or bicyclic ring systems, wherein one or both rings may be saturated or unsaturated, for example they may be aromatic. In particular, bicyclic ring systems will comprise fused 5,6-membered or 6,6-membered rings. Examples of such groups include furyl, thienyl, pyrrolyl, pyrrolidinyl, imidazolyl, triazolyl, thiazolyl, tetrazolyl, oxazolyl, isoxazolyl, pyrazolyl, pyridyl, pyrimidinyl, pyrazinyl, pyridazinyl, triazinyl, indolyl, quinolinyl, isoquinolinyl, quinoxalinyl, benzthiazolyl, benzoxazolyl, benzothienyl or benzofuryl.

When “heterocyclyl” or “heterocyclic” is a mono-cyclic ring, it is for example selected from piperidinyl, piperazinyl, morpholino, thiomorpholino (and versions thereof wherein the sulfur atom is oxidised to SO or SO₂), furyl, thienyl, pyrrolyl, pyrrolidinyl, imidazolyl, triazolyl, thiazolyl, tetrazolyl, oxazolyl, isoxazolyl, pyrazolyl, pyridyl, pyrimidinyl, pyrazinyl, pyridazinyl and triazinyl; and more particularly selected from piperidinyl, piperazinyl, morpholino, thiomorpholino (and versions thereof wherein the sulfur atom is oxidised to SO or SO₂), furyl, thienyl, pyrrolyl, pyrrolidinyl, imidazolyl, thiazolyl, oxazolyl, isoxazolyl, pyrazolyl, pyridyl, pyrimidinyl, pyrazinyl and pyridazinyl.

When “heterocyclyl” or “heterocyclic” is a bi-cyclic ring, it is for example selected from indolyl, quinolinyl, isoquinolinyl, quinoxalinyl, benzthiazolyl, benzoxazolyl, benzothienyl and benzofuryl.

“Heteroaryl” refers to those heterocyclic groups described above which have an aromatic character. Example of heteroaryl mono-cyclic rings include furyl, thienyl, pyrrolyl, imidazolyl, triazolyl, thiazolyl, tetrazolyl, oxazolyl, isoxazolyl, pyrazolyl, pyridyl, pyrimidinyl, pyrazinyl, triazinyl and pyridazinyl; further suitable examples include furyl, thienyl, pyrrolyl, imidazolyl, thiazolyl, oxazolyl, isoxazolyl, pyrazolyl, pyridyl, pyrimidinyl, pyrazinyl and pyridazinyl.

The term “aralkyl” refers to aryl substituted allyl groups such as benzyl.

Other expressions used in the specification include “hydrocarbyl” which refers to any structure comprising carbon and hydrogen atoms. These may be arranged in rings or chains or combinations in which rings are joined to chains or to further rings, or are fused to further rings. Generally, hydrocarbyl groups will contain from 1 to 20, for instance from 1-12 carbon atoms. These may be alkyl, alkenyl, alkynyl, aryl, aralkyl, aralkenyl, aralkynyl, cycloalkyl or cycloalkenyl, wherein any cyclic moiety such as aryl, aralkyl, cycloalkyl or cycloalkenyl are optionally substituted with alkyl, alkenyl, alkynyl and/or with further cyclic moieties, and where any alkyl, alkenyl or alkynyl groups are optionally substituted with cycloalkyl, or cycloalkenyl.

Suitable combinations of rings and chains which are comprised by the term hydrocarbyl include

a) cyclohexyl linked to a (1-6C)alkyl group (in particular cyclohexylmethyl or cyclohexylethyl);

b) cyclohexyl linked to a second cyclohexyl or a cyclopentyl group by a direct bond, or with a (1-6C)alkyl group linker;

c) a phenyl group linked to a second phenyl group by a direct bond, or with a (1-6C)alkyl group linker;

d) a (3-8C)cycloalkylgroup (such as cyclohexyl or cyclopentyl) linked to a phenyl group by a direct bond or with a (1-6C)alkyl linker.

References to a “combination” of hydrocarbyl and heterocyclic groups refer to moieties which contain one or more heterocyclic groups joined to one or more hydrocarbyl groups.

Suitable combinations of hydrocarbyl and heterocyclic groups include a heterocyclyl group (such as morpholino, thiomorpholino, piperazinyl or piperidinyl) linked to a hydrocarbyl group (such as a (1-6C)alkyl group and/or a (3-8C)cycloalkyl group; in particular a (1-6C)alkyl group).

Unless specified otherwise, the expression “haloalkyl” refers to alkyl groups which carry at least one halo substitutent. This includes perhalo groups where all hydrogen atoms are replaced by halo such as fluoro. A similar convention applies to “haloalkoxy”.

It is to be understood that optional substituents on any group may be attached to any available atom as appropriate unless otherwise specified, including heteroatoms provided that they are not thereby quaternised.

Where optional substituents are chosen from “0, 1, 2 or 3” groups it is to be understood that this definition includes all substituents being chosen from one of the specified groups or the substituents being chosen from two or more of the specified groups. An analogous convention applies to substituents chosen from “0, 1 or 2” groups and “1 or 2” and any other analogous groups.

Substituents may be present at any suitable position on, for example, an alkyl group. Therefore, hydroxy substituted (1-6C)alkyl includes hydroxymethyl, 1-hydroxyethyl, 2-hydroxyethyl and 3-hydroxypropyl.

For the avoidance of doubt it is to be understood that where in this specification a group is qualified by ‘hereinbefore defined’ or ‘defined hereinbefore’ the said group encompasses the first occurring and broadest definition as well as each and all of the particular definitions for that group.

It is to be understood that where substituents contain two substituents on an alkyl chain, in which both are linked by a heteroatom (for example two alkoxy substituents), then these two substituents are not substituents on the same carbon atom of the alkyl chain.

If not stated elsewhere, suitable optional substituents for a particular group are those as stated for similar groups herein.

A compound of formula (I) may form stable acid or basic salts, and in such cases administration of a compound as a salt may be appropriate, and pharmaceutically acceptable salts may be made by conventional methods such as those described following.

Suitable pharmaceutically-acceptable salts include acid addition salts such as methanesulfonate, tosylate, α-glycerophosphate, fumarate, hydrochloride, citrate, maleate, tartrate and (less preferably) hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. A preferred pharmaceutically-acceptable salt is the sodium salt.

However, to facilitate isolation of the salt during preparation, salts which are less soluble in the chosen solvent may be preferred whether pharmaceutically-acceptable or not.

Within the present invention it is to be understood that a compound of the formula (I) or a salt thereof may exhibit the phenomenon of tautomerism and that the formulae drawings within this specification can represent only one of the possible tautomeric forms. It is to be understood that the invention encompasses any tautomeric form which inhibits DGAT1 activity and is not to be limited merely to any one tautomeric form utilised within the formulae drawings.

Various forms of prodrugs are known in the art. For examples of such prodrug derivatives, see:

a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and

### H. Bundgaard, Chapter 5 “Design and Application of Prodrugs”, by H. Bundgaard p. 113-191 (1991);

### c) H. Bundgaard, Advanced Drug Delivery Reviews, 8, 1-38 (1992);

### d) H. Bundgaard, et al., Journal of Pharmaceutical Sciences, 77, 285 (1988); and

### e) N. Kakeya, et al., Chem Pharm Bull, 32, 692 (1984).

Examples of such prodrugs are in vivo cleavable esters of a compound of the invention. An in vivo cleavable ester of a compound of the invention containing a carboxy group is, for example, a pharmaceutically-acceptable ester which is cleaved in the human or animal body to produce the parent acid. Suitable pharmaceutically-acceptable esters for carboxy include (1-6C)allyl esters, for example methyl or ethyl; (1-6C)alkoxymethyl esters, for example methoxymethyl; (1-6C)alkanoyloxymethyl esters, for example pivaloyloxymethyl; phthalidyl esters; (3-8C)cycloalkoxycarbonyloxy(1-6C)allyl esters, for example 1-cyclohexylcarbonyloxyethyl; 1,3-dioxolan-2-ylmethyl esters, for example 5-methyl-1,3-dioxolan-2-ylmethyl; (1-6C)alkoxycarbonyloxyethyl esters, for example 1-methoxycarbonyloxyethyl; aminocarbonylmethyl esters and mono- or di-N-((1-6C)allyl) versions thereof, for example N,N-dimethylaminocarbonylmethyl esters and N-ethylaminocarbonylmethyl esters; and may be formed at any carboxy group in the compounds of this invention. An in vivo cleavable ester of a compound of the invention containing a hydroxy group is, for example, a pharmaceutically-acceptable ester which is cleaved in the human or animal body to produce the parent hydroxy group. Suitable pharmaceutically acceptable esters for hydroxy include (1-6C)alkanoyl esters, for example acetyl esters; and benzoyl esters wherein the phenyl group may be substituted with aminomethyl or N-substituted mono- or di-(1-6C)alkyl aminomethyl, for example 4-aminomethylbenzoyl esters and 4-N,N-dimethylaminomethylbenzoyl esters.

It will be appreciated by those skilled in the art that certain compounds of formula (I) contain asymmetrically substituted carbon and/or sulfur atoms, and accordingly may exist in, and be isolated in, optically-active and racemic forms. Some compounds may exhibit polymorphism. It is to be understood that the present invention encompasses any racemic, optically-active, polymorphic or stereoisomeric form, or mixtures thereof, which form possesses properties useful in the inhibition of DGAT1 activity, it being well known in the art how to prepare optically-active forms (for example, by resolution of the racemic form by recrystallization techniques, by synthesis from optically-active starting materials, by chiral synthesis, by enzymatic resolution, by biotransformation, or by chromatographic separation using a chiral stationary phase) and how to determine efficacy for the inhibition of DGAT1 activity by the standard tests described hereinafter.

It is also to be understood that certain compounds of the formula (I) and salts thereof can exist in solvated as well as unsolvated forms such as, for example, hydrated forms. It is to be understood that the invention encompasses all such solvated forms which inhibit DGAT1 activity.

As stated before, we have discovered a range of compounds that have good DGAT1 inhibitory activity. They have good physical and/or pharmacokinetic properties in general. The following compounds possess preferred pharmaceutical and/or physical and/or pharmacokinetic properties.

Particular aspects of the invention comprise a compound of formula (I), or a salt (particularly a pharmaceutically-acceptable) thereof, wherein any of the groups/substituents mentioned above have values defined hereinbefore, or any of the following values (which may be used where appropriate with any of the definitions and embodiments disclosed hereinbefore or hereinafter):

In one embodiment of the invention are provided compounds of formula (I), in an alternative embodiment are provided salts (particularly pharmaceutically-acceptable salts) of compounds of formula (I). In a further embodiment are provided pro-drugs of compounds of formula (I). In a still further embodiment are provided salts, particularly pharmaceutically-acceptable salts of pro-drugs of compounds of formula (I).

In one aspect, R¹ is an optionally substituted aryl group such as optionally substituted phenyl or napthyl.

In another aspect, R¹ is an optionally substituted heteroaryl group, for example an optionally substituted monocyclic heteroaryl group such as pyridyl, thienyl or isoxazolyl, or an optionally substituted bicyclic heteroaryl group such as indolyl, quinoxalinyl, benzothienyl or benzofuryl.

In a further aspect, R¹ is selected from an optionally substituted phenyl, naphthyl, thienyl, isoxazolyl, indolyl, benzothienyl, benzofuryl and quinoxalinyl.

Suitable optional substitutents for R¹ include groups independently selected from R^{f} or (1-6C)alkyl groups such as methyl, ethyl or tert-butyl. Particular values of R^{f} for substituents on R¹ include halo, nitro, cyano, C(O)ₙR¹⁴ or OR¹⁴, where R¹⁴ is as defined above, and in particular is an aryl (such as phenyl), aralkyl (such as benzyl) or (1-6C)alkyl optionally substituted with halo (such as methyl, isopropyl and difluoromethyl) group.

Where R¹ is substituted by a group —X²—(CR³R⁴)_{q}-Z^{a}, a group —X²—(CR³R⁴)ₐ—X³-Z^{a} or a group —(CR³R⁴)ᵥX³-Z^{a}, R³ and R⁴ are suitably hydrogen.

Further suitable substituents for R¹ include halo (such as fluoro or chloro), (1-4C)alkyl, (1-4C)alkoxy, benzyloxy, cyano, nitro and halo(1-4C)alkoxy (such as difluoromethoxy).

W is selected from —C(O)—, —C(O)O—, —C(O)NH— and —C(O)(CR^{A}R^{B})ₖ—;

In one aspect W is —C(O)—.

In another aspect, W is —C(O)(CR^{A}R^{B})ₖ—. In this aspect, suitably k is 1 and R^{A} and R^{B} are independently hydrogen or methyl. In another embodiment of this aspect, k is 1 and R^{A} and R^{B} together form a cyclobutyl, cyclopentyl or cyclohexyl ring.

Suitably W is selected from —C(O)—, —C(O)CH₂—, —C(O)CH(Me)—, —C(O)C(Me)₂-, —C(O)CR^{A}R^{B}— (wherein R^{A} and R^{B} together form a cyclopropyl, cyclobutyl, cyclopentyl or cyclohexyl ring).

In a preferred embodiment, Y is a direct bond.

Where Y is a group —X⁶(CR⁵R⁶)ₜ, X⁶ is suitably oxygen and t is preferably an integer of from 2 to 6.

Alternatively, Y is a group (CH₂), or more preferably —O(CH₂)ₜ— where s is an integer of from 1 to 6 and t is an integer of from 2 to 6, and in particular s or t are 3.

When R² is unsubstituted aryl, unsubstituted (3-8C)cycloalkyl, unsubstituted (5-12C)bicycloalkyl, or unsubstituted (6-12C)tricycloalkyl, Y is preferably other than a direct bond.

R² is a suitably a substituted phenyl or a substituted heteroaryl group.

In another embodiment, R² is a suitably a substituted (3-8C)cycloalkyl (particularly cyclohexyl), a substituted (5-12C)bicycloalkyl (such as norbornyl) or a substituted (6-12C)tricycloalkyl (such as adamantyl).

When R² is a substituted group, it is suitably substituted by at least one and optionally more than one substitutent group -Z, a group —X—(CR⁷R⁸)ᵤ-Z, a group —X—(CR⁷R⁸)ᵥ—X¹-Z or a group —(CR⁷R⁸)ᵥX¹-Z, where one or more further substituents may be selected from halo, cyano, nitro, amino, hydroxy or halo(1-6C)alkyl. In one embodiment, R² is substituted by Z.

Particular examples of groups Z or Z^{a} include groups of sub formula (x), (y) or (z)

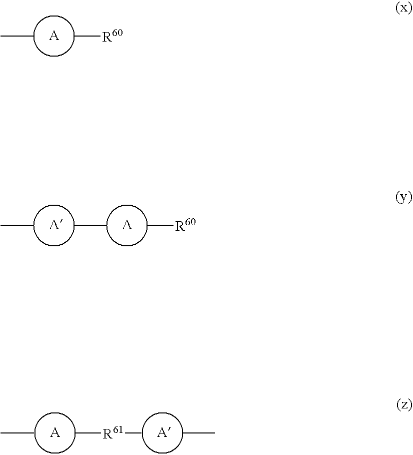

wherein each ring A or A′ is independently selected from an optionally substituted heterocyclic ring, an optionally substituted cycloalkyl ring or an optionally substituted aryl ring, each R⁶⁰ is an optionally substituted (1-6C)alkyl, an optionally substituted (2-6C)alkenyl or an optionally substituted (2-6C)alkynyl, and R⁶¹ is an optionally substituted (1-6C)alkylene, an optionally substituted (2-6C)alkenylene or an optionally substituted (2-6C)alkynylene.

Suitably, optional substituents for groups A, A′, R⁶⁰ and R⁶¹ are groups independently selected at each occurrence from R^{f}.

In one aspect, Z is a group of sub-formula (x) above. In one embodiment of this aspect, ring A is selected from morpholino, piperazinyl (particularly N-acetylpiperazinyl) and cyclohexyl (optionally substituted, suitably with a (1-4C) alkyl group substituted with carboxyalkyl or the methylester thereof).

In one embodiment, R² is a 5- or 6-membered aromatic ring of sub-structure (a)

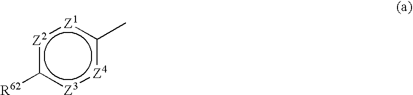

Z¹, Z², Z³ and Z⁴ are independently selected from —CH—, —CR^{z}— or a heteroatom selected from O, S, N(R⁵⁰)ᵣ, where r is 0 or 1 depending upon the requirements of the aromatic ring, and R⁵⁰ is hydrogen or C₁₋₆alkyl, and Z⁴ may additionally be a direct bond,

R⁶² is a group -Z, a group —X—(CR⁷R⁸)ᵤ-Z, a group —X—(CR⁷R⁸)ᵥ—X¹-Z or a group —(CR⁷R⁸)ᵥX¹-Z, wherein Z, X, X¹ R⁷, R⁸, u and v are as defined above,

each R^{z} is independently selected from halo, cyano, nitro, amino, hydroxy, halo(1-6C)allyl, a group -Z, a group —X—(CR⁷R⁸)ᵤ-Z, a group —X—(CR⁷R⁸)ᵥ—X¹-Z or a group —(CR⁷R⁸)ᵥX¹-Z, wherein Z, X, X¹ R⁷, R⁸, u and v are as defined above.

Suitably, when Z⁴ is a direct bond, one of Z¹ or Z² is a heteroatom, in particular oxygen or sulphur.

Preferably Z⁴ is other than a direct bond.

Suitably in this case, Z² and Z³ are independently selected from —CH—, —CR^{z}— or a nitrogen atom.

Suitably Z¹ is a —CH— group.

Suitably, Z¹, Z², Z³ and Z⁴ are —CH—.

Suitably R⁷ and R⁸ are hydrogen.

In one aspect R^{z} is selected from halo, cyano, nitro, amino, hydroxy and halo(1-6C)alkyl. Suitably, R^{z} is halo, such as fluoro. in another aspect, R^{z} is selected from a group -Z, a group —X—(CR⁷R⁸)ᵤ-Z, a group —X—(CR⁷R⁸)ᵥ—X¹-Z or a group —(CR⁷R⁸)ᵥX¹-Z

In an alternative embodiment, R² is a cycloalkyl group such as cyclohexyl of sub-formula (b)

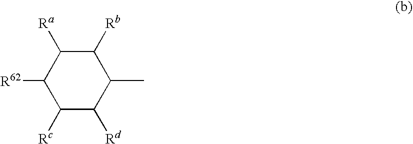

where R⁶² is as defined above, and R^{a}, R^{b}, R^{c} and R^{d} are independently selected from hydrogen or a group R^{z} as defined above. In one embodiment, R^{a}, R^{b}, R^{c} and R^{d} are all hydrogen.

In yet a further embodiment, R² is a bicyclic ring, which may be a bicyclic aryl ring or a bicyclic heterocyclic ring. For instance, R² comprises fused 6,6-membered rings, or fused 5,6-membered rings, one or both of said rings may be unsaturated. Examples of such rings include benzimidazole (preferably linked to the group-Y—NH— by way of the benzene ring), indanyl, indenyl. Particularly suitable bicyclic rings are partially unsaturated, such that the ring linked to the group-Y—NH— is saturated and this is fused to an aromatic ring. Particular examples of such rings are indanyl rings, such as 2-indanyl.

In particular, R⁶² in subformula (a) or (b) is a group Z. Suitably Z is an aryl, heterocyclyl or cycloalkyl group, any of which are optionally substituted by a group independently selected from R^{f} or an (1-6C)alkyl, (2-6C)alkenyl or (2-6C)alkynyl group. Further, Z is suitably phenyl or phenyl(1-6C)alkyl (such as benzyl).

In another aspect, Z is a heterocyclyl group, optionally substituted by a group independently selected from R^{f} or an (1-6C)allyl, (2-6C)alkenyl or (2-6C)alkynyl group. In this aspect, Z is suitably selected from morpholino, thiomorpholino, piperidinyl and N-substituted-piperazino; particularly, Z is morpholino or N-acetylpiperazino.

Preferably Z is substituted by a group selected from R^{f} or by a (1-6C) alkyl group which is substituted by a group selected from R^{f}. Particular examples of such groups selected from R^{f} include C(O)₂R¹⁴ or a carboxylic acid mimic or bioisostere thereof, C(O)NR¹⁵R¹⁶ or —NR¹⁵C(O)ₙR¹⁴, where R¹⁴, R¹⁵ and R¹⁶ are as defined above.

In one aspect, R² is selected from optionally substituted phenyl and pyridyl, particularly phenyl and 3-pyridyl (with respect to the bond to the amide nitrogen). Suitable optional substituents in this aspect include fluoro, and/or one substituent selected from morpholino, N-acylpiperazine, 3-(carboxymethyl)cyclohexyl and 3-(methoxycarbonylmethyl)cyclohexyl.

In one aspect of the invention, there is provided a compound of formula (I), or a salt or prodrug thereof, which is a compound of formula (IA):

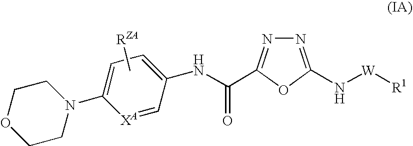

wherein X^{A} is CH or N, R^{ZA} is halo, particularly fluoro, and W and R¹ are as hereinbefore defined for a compound of formula (I) in any aspect or embodiment. Suitably W is selected from —C(O)—, —C(O)CH₂—, —C(O)CH(Me)—, —C(O)C(Me)₂-, —C(O)CR^{A}R^{B}— (wherein R^{A} and R^{B} together form a cyclopropyl, cyclobutyl, cyclopentyl or cyclohexyl ring); and R¹ is selected from an optionally substituted phenyl, naphthyl, thienyl, isoxazolyl, indolyl, benzothienyl, benzofuryl and quinoxalinyl, wherein suitable optional substituents for R¹ include halo (such as fluoro or chloro), (1-4C)alkyl, (1-4C)alkoxy, benzyloxy, cyano, nitro and halo(1-4C)alkoxy (such as difluoromethoxy).

In one aspect of the invention, there is provided a compound of formula (I), or a salt or prodrug thereof, which is a compound of formula (IB):

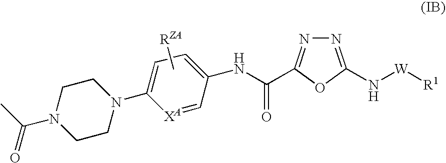

wherein X^{A} is CH or N (particularly CH), R^{ZA} is halo (particularly fluoro), and W and R¹ are as hereinbefore defined for a compound of formula (I) in any aspect or embodiment. Suitably W is selected from —C(O)—, —C(O)CH₂—, —C(O)CH(Me)—, —C(O)C(Me)₂-, —C(O)CR^{A}R^{B}— (wherein R^{A} and R^{B} together form a cyclopropyl, cyclobutyl, cyclopentyl or cyclohexyl ring); and R¹ is selected from an optionally substituted phenyl, naphthyl, thienyl, isoxazolyl, indolyl, benzothienyl, benzofuryl and quinoxalinyl, wherein suitable optional substituents for R¹ include halo (such as fluoro or chloro), (1-4C)alkyl, (1-4C)alkoxy, benzyloxy, cyano, nitro and halo(1-4C)alkoxy (such as difluoromethoxy).

In one aspect of the invention, there is provided a compound of formula (I), or a salt or prodrug thereof, which is a compound of formula (IC):

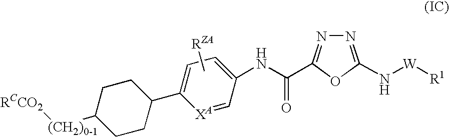

wherein X^{A} is CH or N (particularly CH), R^{ZA} is halo (particularly fluoro), R^{C} is hydrogen or methyl (particularly hydrogen) and W and R¹ are as hereinbefore defined for a compound of formula (I) in any aspect or embodiment. Suitably W is selected from —C(O)—, —C(O)CH₂—, —C(O)CH(Me)—, —C(O)C(Me)₂-, —C(O)CR^{A}R^{B}— (wherein R^{A} and R^{B} together form a cyclopropyl, cyclobutyl, cyclopentyl or cyclohexyl ring); and R¹ is selected from an optionally substituted phenyl, naphthyl, thienyl, isoxazolyl, indolyl, benzothienyl, benzofuryl and quinoxalinyl, wherein suitable optional substituents for R¹ include halo (such as fluoro or chloro), (1-4C)alkyl, (1-4C)alkoxy, benzyloxy, cyano, nitro and halo(1-4C)alkoxy (such as difluoromethoxy).

Reference herein to a compound of formula (I) should be taken to apply equally to a compound of formula (IA), (IB) and/or (IC), unless the context requires otherwise.

When R² is phenyl substituted by acylpiperazine (such as a compound of formula (IB)), Y is a direct bond and W is C(O), then preferably R¹ is not 2-ethoxyphenyl or 2-chlorophenyl. When R² is phenyl substituted by acylpiperazine (such as a compound of formula (IB)), and W is C(O), then in one aspect, R¹ is not substituted in either of the 2-positions (relative to the point of attachment to W).

When W is C(O), in one aspect, R¹ is not substituted in either of the 2-positions (relative to the point of attachment to W).

As used herein, the reference to carboxylic acid mimic or bioisostere includes groups as defined in The Practice of Medicinal Chemistry, Wermuth C. G. Ed.: Academic Press: New York, 1996, p203. Particular examples of such groups include —SO₃H, S(O)₂NHR¹³, —S(O)₂NHC(O)R¹³, —CH₂S(O)₂R¹³, —C(O)NHS(O)₂R¹³, —C(O)NHOH, —C(O)NHCN, —CH(CF₃)OH, C(CF₃)₂OH, —P(O)(OH)₂ and groups of sub-formula (a)-(i′) below

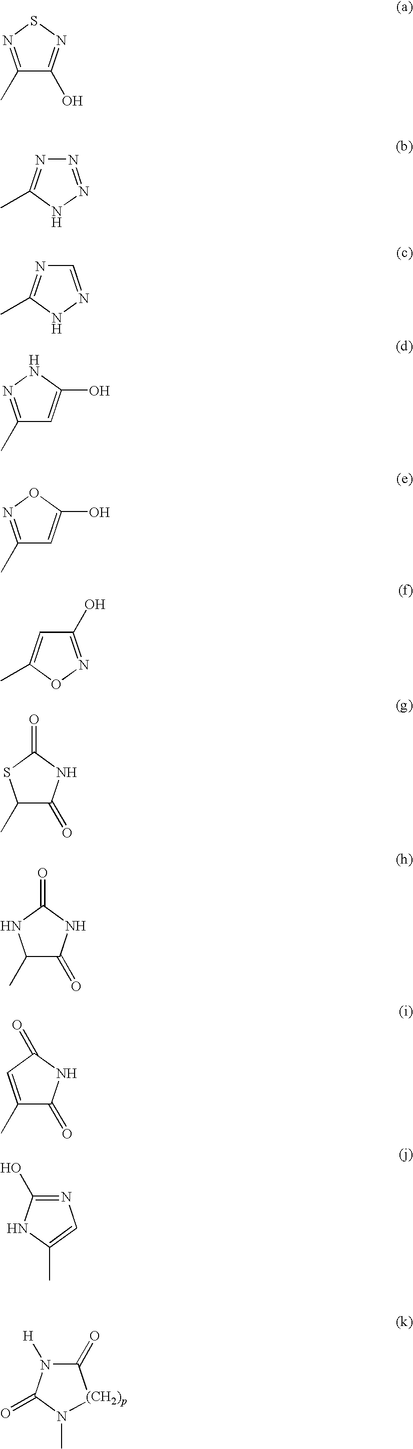

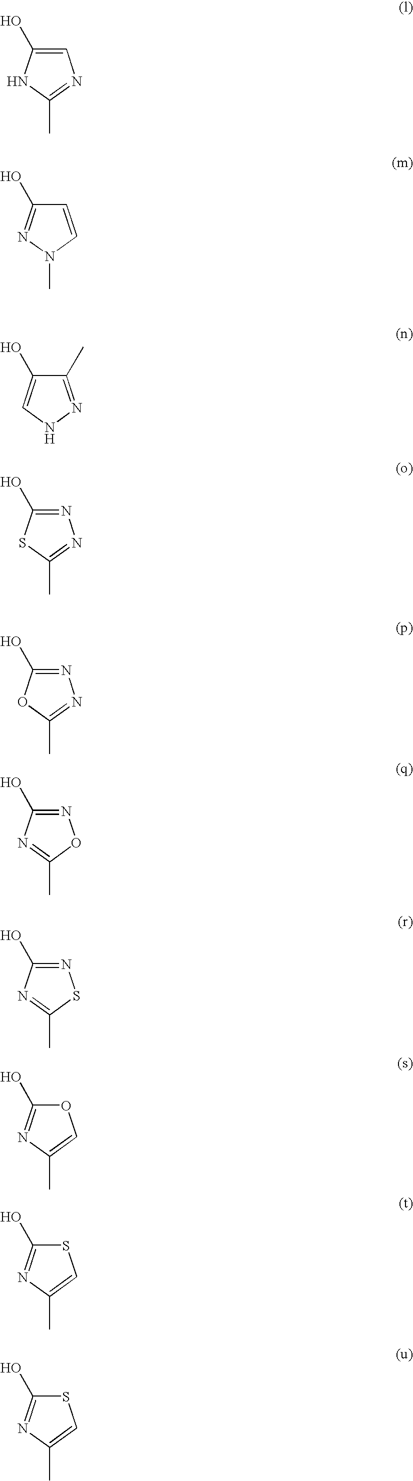

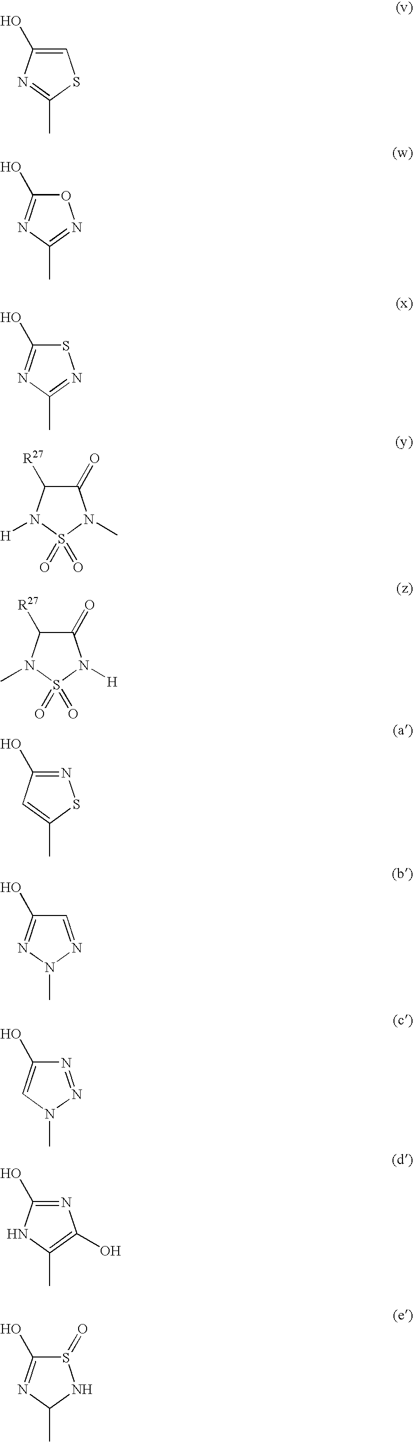

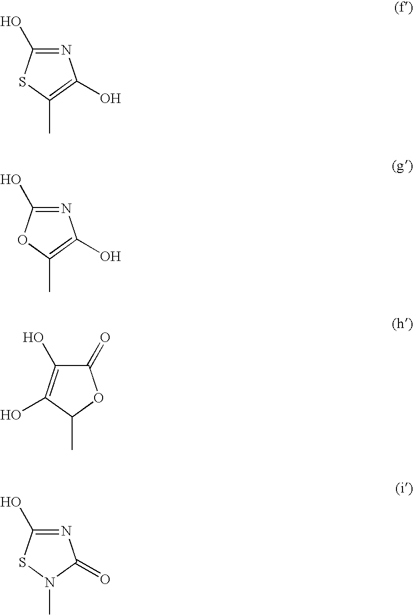

wherein R¹³ is (1-6C)alkyl, aryl or heteroaryl; and R²⁷ is hydrogen or (1-4C)alkyl. It will be understood that in the above sub-formulae (a) to (i′), keto-enol tautomerism may be possible and that the sub-formulae (a) to (i′) should be taken to encompass all tautomers thereof.

Preferred compounds of the invention are each of the Examples, or a pharmaceutically-acceptable salt, or pro-drug thereof, each of which provides a further independent aspect of the invention. In further aspects, the present invention also comprises any two or more compounds of the Examples or a pharmaceutically-acceptable salt, or pro-drug thereof.

Preferred compounds of the invention are any one of the following, or their salts (particularly pharmaceutically acceptable salts) or pro-drugs:
5-[(4-chlorobenzoyl)amino]-N-(3-fluoro-4-morpholin-4-ylphenyl)-1,3,4-oxadiazole-2-carboxamide;
5-{[2-(4-chlorophenyl)-2-methylpropanoyl]amino}-N-(4-morpholin-4-ylphenyl)-1,3,4-oxadiazole-2-carboxamide;
5-[(4-chlorobenzoyl)amino]-N-(6-morpholin-4-ylpyridin-3-yl)-1,3,4-oxadiazole-2-carboxamide;
methyl (trans-4-{4-[({5-[(4-chlorobenzoyl)amino]-1,3,4-oxadiazol-2-yl}carbonyl)amino]phenyl}cyclohexyl)acetate;
(trans-4-{4-[({5-[(4-chlorobenzoyl)amino]-1,3,4-oxadiazol-2-yl}carbonyl)amino]phenyl}cyclohexyl)acetic acid;
5-(benzoylamino)-N-(4-morpholin-4-ylphenyl)-1,3,4-oxadiazole-2-carboxamide;
5-[(3-fluorobenzoyl)amino]-N-(4-morpholin-4-ylphenyl)-1,3,4-oxadiazole-2-carboxamide;
5-[(3-methylbenzoyl)amino]-N-(4-morpholin-4-ylphenyl)-1,3,4-oxadiazole-2-carboxamide;
5-[(4-fluorobenzoyl)amino]-N-(4-morpholin-4-ylphenyl)-1,3,4-oxadiazole-2-carboxamide;
5-[(4-methoxybenzoyl)amino]-N-(4-morpholin-4-ylphenyl)-1,3,4-oxadiazole-2-carboxamide;
5-[(4-methylbenzoyl)amino]-N-(4-morpholin-4-ylphenyl)-1,3,4-oxadiazole-2-carboxamide;
5-[(4-cyanobenzoyl)amino]-N-(4-morpholin-4-ylphenyl)-1,3,4-oxadiazole-2-carboxamide;
N-(4-morpholin-4-ylphenyl)-5-(1-naphthoylamino)-1,3,4-oxadiazole-2-carboxamide;
N-(4-morpholin-4-ylphenyl)-5-(2-naphthoylamino)-1,3,4-oxadiazole-2-carboxamide;
N-(4-morpholin-4-ylphenyl)-5-[(4-nitrobenzoyl)amino]-1,3,4-oxadiazole-2-carboxamide;
N-(4-morpholin-4-ylphenyl)-5-[(phenylacetyl)amino]-1,3,4-oxadiazole-2-carboxamide;
N-(6-morpholin-4-ylpyridin-3-yl)-5-[(phenylacetyl)amino]-1,3,4-oxadiazole-2-carboxamide;
N-[4-(4-acetylpiperazin-1-yl)phenyl]-5-[(4-isopropoxybenzoyl)amino]-1,3,4-oxadiazole-2-carboxamide;
N-[4-(4-acetylpiperazin-1-yl)phenyl]-5-[(4-methylbenzoyl)amino]-1,3,4-oxadiazole-2-carboxamide;
N-[4-(4-acetylpiperazin-1-yl)phenyl]-5-[(4-chlorobenzoyl)amino]-1,3,4-oxadiazole-2-carboxamide;
N-[4-(4-acetylpiperazin-1-yl)phenyl]-5-[(4-tert-butylbenzoyl)amino]-1,3,4-oxadiazole-2-carboxamide;
N-[4-(4-acetylpiperazin-1-yl)phenyl]-5-[(4-methoxybenzoyl)amino]-1,3,4-oxadiazole-2-carboxamide;
N-[4-(4-acetylpiperazin-1-yl)phenyl]-5-{[4-(difluoromethoxy)benzoyl]amino}-1,3,4-oxadiazole-2-carboxamide;
5-{[2-(4-chlorophenyl)-2-methylpropanoyl]amino}-N-(4-morpholin-4-ylphenyl)-1,3,4-oxadiazole-2-carboxamide;
5-({[1-(2,4-dichlorophenyl)cyclopropyl]carbonyl}amino)-N-(4-morpholin-4-ylphenyl)-1,3,4-oxadiazole-2-carboxamide;
5-({[1-(4-chlorophenyl)cyclobutyl]carbonyl}amino)-N-(4-morpholin-4-ylphenyl)-1,3,4-oxadiazole-2-carboxamide;
5-({[1-(4-chlorophenyl)cyclopentyl]carbonyl}amino)-N-(4-morpholin-4-ylphenyl)-1,3,4-oxadiazole-2-carboxamide;
N-(4-morpholin-4-ylphenyl)-5-{[(1-phenylcyclopentyl)carbonyl]amino}-1,3,4-oxadiazole-2-carboxamide;
5-{[2-(4-chlorophenyl)-2-methylpropanoyl]amino}-N-(6-morpholin-4-ylpyridin-3-yl)-1,3,4-oxadiazole-2-carboxamide;
N-(6-morpholin-4-ylpyridin-3-yl)-5-{[(1-phenylcyclopentyl)carbonyl]amino}-1,3,4-oxadiazole-2-carboxamide;
5-({[1-(3-fluorophenyl)cyclopentyl]carbonyl}amino)-N-(6-morpholin-4-ylpyridin-3-yl)-1,3,4-oxadiazole-2-carboxamide;
5-({[1-(2-fluorophenyl)cyclopentyl]carbonyl}amino)-N-(6-morpholin-4-ylpyridin-3-yl)-1,3,4-oxadiazole-2-carboxamide;
N-[4-(4-acetylpiperazin-1-yl)phenyl]-5-[(4-benzyloxybenzoyl)amino]-1,3,4-oxadiazole-2-carboxamide;
N-[4-(4-acetylpiperazin-1-yl)phenyl]-5-[(3-isobutoxybenzoyl)amino]-1,3,4-oxadiazole-2-carboxamide;
N-[4-(4-acetylpiperazin-1-yl)phenyl]-5-[(3-isopropoxybenzoyl)amino]-1,3,4-oxadiazole-2-carboxamide;
N-[4-(4-acetylpiperazin-1-yl)phenyl]-5-[(2-ethylbenzoyl)amino]-1,3,4-oxadiazole-2-carboxamide;
N-[4-(4-acetylpiperazin-1-yl)phenyl]-5-{[3-(difluoromethoxy)benzoyl]amino}-1,3,4-oxadiazole-2-carboxamide;
N-[4-(4-acetylpiperazin-1-yl)phenyl]-5-({[1-(4-chlorophenyl)cyclohexyl]carbonyl}amino)-1,3,4-oxadiazole-2-carboxamide;
N-[4-(4-acetylpiperazin-1-yl)phenyl]-5-({[1-(4-chlorophenyl)cyclopentyl]carbonyl}amino)-1,3,4-oxadiazole-2-carboxamide;
N-[4-(4-acetylpiperazin-1-yl)phenyl]-5-({[1-(4-chlorophenyl)cyclobutyl]carbonyl}amino)-1,3,4-oxadiazole-2-carboxamide;
N-[4-(4-acetylpiperazin-1-yl)phenyl]-5-({[1-(3-fluorophenyl)cyclopentyl]carbonyl}amino)-1,3,4-oxadiazole-2-carboxamide;
N-[4-(4-acetylpiperazin-1-yl)phenyl]-5-({[1-(2-fluorophenyl)cyclopentyl]carbonyl}amino)-1,3,4-oxadiazole-2-carboxamide;
N-[4-(4-acetylpiperazin-1-yl)phenyl]-5-({[1-(4-fluorophenyl)cyclopentyl]carbonyl}amino)-1,3,4-oxadiazole-2-carboxamide;
N-[4-(4-acetylpiperazin-1-yl)phenyl]-5-{[(1-phenylcyclopentyl)carbonyl]amino}-1,3,4-oxadiazole-2-carboxamide;
N-[4-(4-acetylpiperazin-1-yl)phenyl]-5-{[2-(4-chlorophenyl)-2-methylpropanoyl]amino}-1,3,4-oxadiazole-2-carboxamide;
N-[4-(4-acetylpiperazin-1-yl)phenyl]-5-({[1-(4-methoxyphenyl)cyclopentyl]carbonyl}amino)-1,3,4-oxadiazole-2-carboxamide;
N-[4-(4-acetylpiperazin-1-yl)phenyl]-5-({[1-(4-chlorophenyl)cyclopropyl]carbonyl}amino)-1,3,4-oxadiazole-2-carboxamide;
N-[4-(4-acetylpiperazin-1-yl)phenyl]-5-{[(1-phenylcyclopropyl)carbonyl]amino}-1,3,4-oxadiazole-2-carboxamide;
N-[4-(4-acetylpiperazin-1-yl)phenyl]-5-{[(2S)-2-phenylpropanoyl]amino}-1,3,4-oxadiazole-2-carboxamide;
N-[4-(4-acetylpiperazin-1-yl)phenyl]-5-({[1-(4-methoxyphenyl)cyclopropyl]carbonyl}amino)-1,3,4-oxadiazole-2-carboxamide;
N-[4-(4-acetylpiperazin-1-yl)phenyl]-5-[(2-thienylacetyl)amino]-1,3,4-oxadiazole-2-carboxamide;
N-[4-(4-acetylpiperazin-1-yl)phenyl]-5-[(3-thienylacetyl)amino]-1,3,4-oxadiazole-2-carboxamide;
N-[4-(4-acetylpiperazin-1-yl)phenyl]-5-{[(1-methyl-1H-indol-3-yl)acetyl]amino}-1,3,4-oxadiazole-2-carboxamide;
N-[4-(4-acetylpiperazin-1-yl)phenyl]-5-[(1-benzothien-3-ylacetyl)amino]-1,3,4-oxadiazole-2-carboxamide;
N-[4-(4-acetylpiperazin-1-yl)phenyl]-5-[(1-benzothien-2-ylcarbonyl)amino]-1,3,4-oxadiazole-2-carboxamide;
N-[4-(4-acetylpiperazin-1-yl)phenyl]-5-{[(5-methylisoxazol-3-yl)carbonyl]amino}-1,3,4-oxadiazole-2-carboxamide;
N-[4-(4-acetylpiperazin-1-yl)phenyl]-5-[(2-thienylcarbonyl)amino]-1,3,4-oxadiazole-2-carboxamide;
N-[4-(4-acetylpiperazin-1-yl)phenyl]-5-{[(5-methyl-2-thienyl)carbonyl]amino}-1,3,4-oxadiazole-2-carboxamide;
N-[4-(4-acetylpiperazin-1-yl)phenyl]-5-[(1-benzofuran-2-ylcarbonyl)amino]-1,3,4-oxadiazole-2-carboxamide; and
N-[5-({[4-(4-acetylpiperazin-1-yl)phenyl]amino}carbonyl)-1,3,4-oxadiazol-2-yl]quinoxaline-2-carboxamide.

### Process

A compound of formula (I) and its pharmaceutically-acceptable salts may be prepared by any process known to be applicable to the preparation of chemically related compounds. Such processes, when used to prepare a compound of the formula (I), or a pharmaceutically-acceptable salt thereof, are provided as a further feature of the invention.

In a further aspect the present invention also provides that the compounds of the formula (I) and pharmaceutically-acceptable salts or prodrugs thereof, can be prepared by a process a) to c) as follows (wherein all variables are as hereinbefore defined for a compound of formula (I) unless otherwise stated):

a) reaction of a compound of formula (I) to form another compound of formula (I);

b) where Y is not a direct bond or where R² is not aromatic, by reaction of an amine of formula (2) with a carboxylate salt of formula (3);

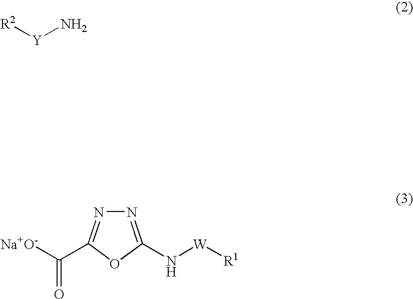

c) cyclisation of a compound of formula (4) (wherein X is O or S);

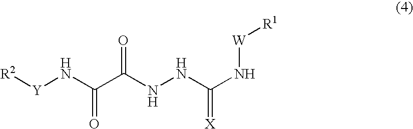

and thereafter if necessary:

i) removing any protecting groups;

ii) forming a salt; and/or

iii) forming a prodrug thereof.

### Process a)

Examples of conversions of a compound of formula (I) into another compound of formula (I), well known to those skilled in the art, include functional group interconversions such as hydrolysis (in particular ester hydrolysis), oxidation or reduction (such as the reduction of an acid to an alcohol, or removal of an N protecting group), and/or further functionalisation by standard reactions such as amide or metal-catalysed coupling, or nucleophilic displacement reactions.

### Process b)

Compounds of formula (2) where Y is not a direct bond or where R² is not aromatic may be made by application of standard synthetic methods well known in the art. For example, reductive allylation of ammonia (or a suitable amine such as a benzylamine or N,N-dibenzylamine) with a ketone or aldehyde R²Y═O (followed by deprotection as appropriate) provides R²—Y—NH₂. Alternatively, alkylation of an amine or amine equivalent (such as a Gabriel reagent or a guanidine) with a halide R²—Y—X (where X is a halide) (followed by N-deprotection or hydrolysis as appropriate) provides the required compounds of formula (2).

Compounds of formula (2) for other definitions of Y or R² may be made by metal catalysed couplings or nucleophilic displacement reactions among other methods. In particular, such compounds of formula (2) may be prepared by reduction of a compound of formula (2A):

R²—Y—NO₂ (2A)

Compounds of formula (2A) may be made by metal catalysed couplings or nucleophilic displacement reactions depending upon the nature of the R² group and Y. For example, production of a compound of formula (2A) may be represented as follows:

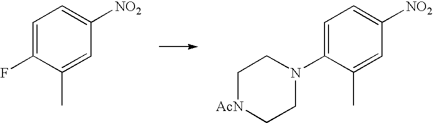

Examples of synthesis of compounds of formula (2) where Y is a direct bond are shown in Schemes 1 to 3:

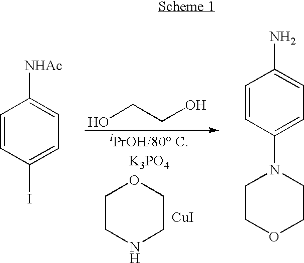

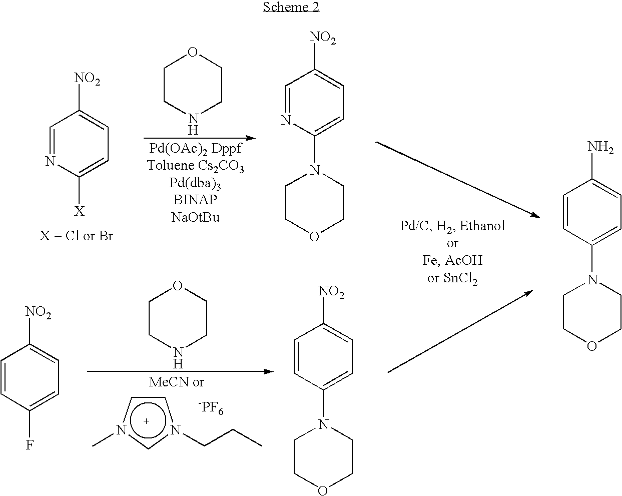

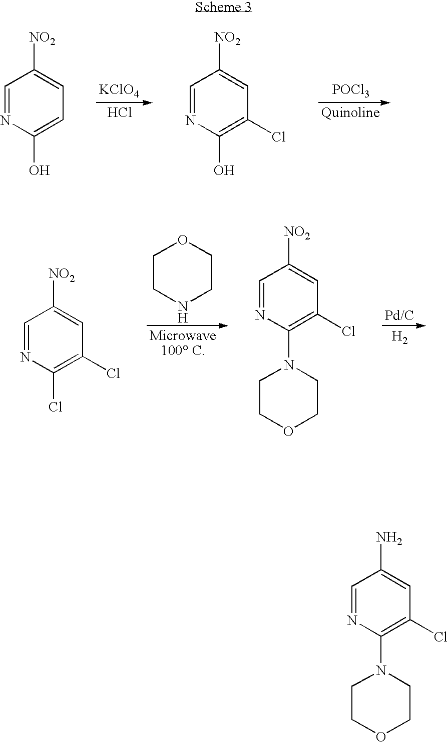

It will be appreciated that the reactions of Schemes 1-3 apply to compounds of formula (2) wherein the phenyl or pyridyl ring is further substituted, for example with halo.

Certain compounds of formula (2) may also have chiral centres or can exist in different isomeric forms such as cis/trans isomers, and may be prepared as individual isomers, as illustrated below in Schemes 4 and 5.

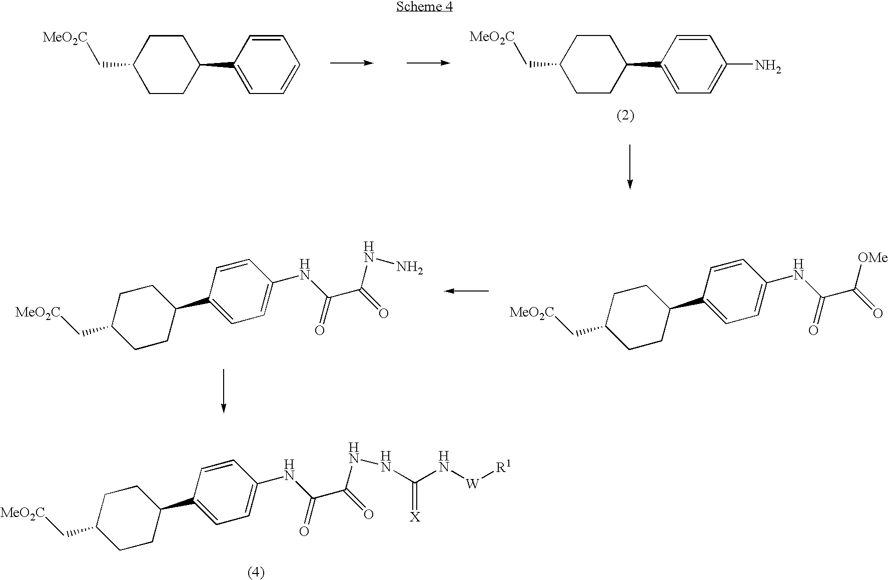

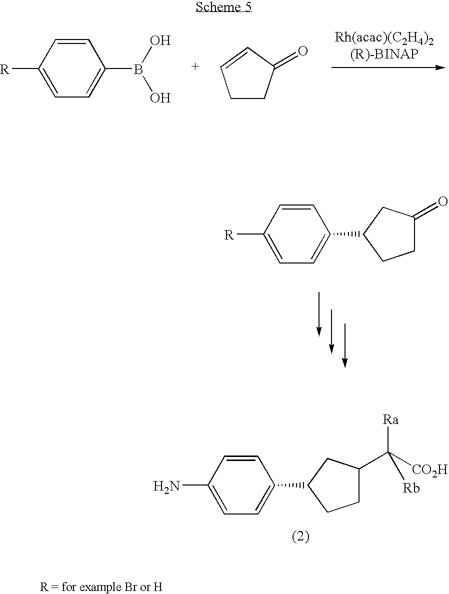

The process illustrated in Scheme 5 may also be used with cyclohexenone as a starting material. The opposite stereochemistry may be obtained by using known alternative chiral catalysts and/or chiral ligands. Elaboration of the bicyclic ketone intermediate may be carried out by processes known in the art, for example by Wittig or enolate/enol ether chemistry, optionally followed by functionalisation (such as alkylation) and functional group interconversion as desired to give the compound of formula (2) (wherein Ra and Rb may each for example be hydrogen or (optionally substituted) alkyl groups). Mixtures of diastereoisomers may be separated by standard procedures.

S_{N}Ar chemistry may be used (under conditions well known in the art) to make certain compounds of formula (2), as illustrated in Scheme 6 (in which R is for example an allyl group, X is for example Br or Cl, n is for example 0 to 4, group A may be a (hetero)aryl ring, a saturated ring or an alkyl chain).

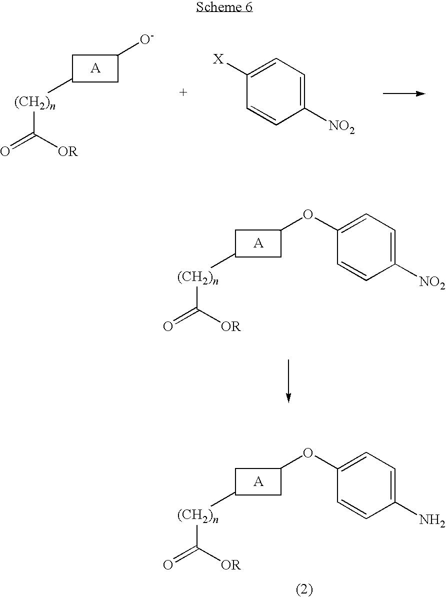

Compounds of formula (3) may be made by alkaline hydrolysis of ester (5a) as prepared using a published procedure (J. Het. Chem. 1977, 14, 1385-1388) or cyclisation of a compound of formula (5b) (where X is O or S) in a similar manner as described in process c) for compounds of formula (4).

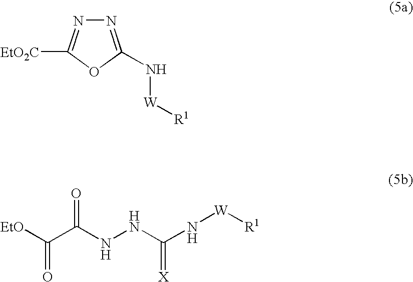

Compounds of formula (2) where Y is not a direct bond or where R² is not aromatic may be coupled with compounds of formula (3) under standard conditions for formation of amide bonds. For example using an appropriate coupling reaction, such as a carbodiimide coupling reaction performed with EDAC, optionally in the presence of DMAP, in a suitable solvent such as DCM, chloroform or DMF at room temperature.

For compounds of formula (2) other than when R² is aromatic and Y is a direct bond (ie other than compounds such as anilino compounds), an ester derivative of formula (5a) (or equivalent) may be used instead of the compound of formula (3) to couple with the compound of formula (2). Such a reaction may be carried out by any method known in the art such as by heating (thermally or by microwave) in a suitable solvent.

### Process c)

Compounds of formula (4) and (5b) where X is S may be made by reaction of an aminocarbonyl acylhydrazine or ethoxycarbonyl acylhydrazine with a thioisocyanate or thioisocyanate equivalent such as aminothiocarbonylimidazole in a suitable solvent such as DMF or MeCN at a temperature between 0 and 100° C. The preparation of aminocarbonyl acylhydrazines from anilines and of ethoxycarbonyl acylhydrazines is well known in the art. For example reaction of an aniline with methyl chlorooxoacetate in the presence of pyridine in a suitable solvent such as DCM followed by reaction with hydrazine in a suitable solvent such as ethanol at a temperature between 0 and 100° C.

The compound of formula (4) may then be cyclised using, for example agents such as carbonyldiimidazole, or tosyl chloride and a suitable base (such as triethylamine), under conditions known in the art.

An example of process c) is shown in Scheme 7:

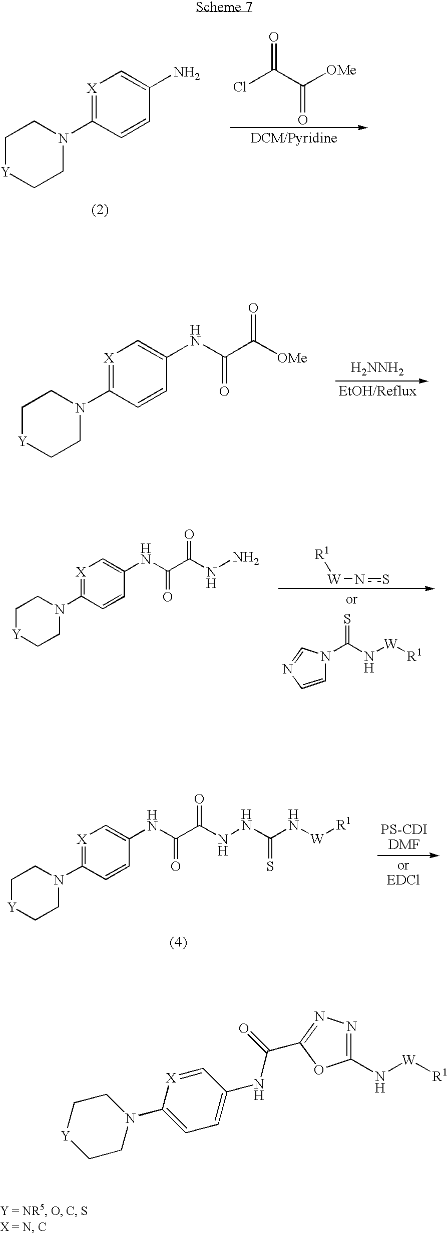

Iso(thio)cyanates R¹—W—NCX (where X is O or S) are commercially available or may be made by reaction of the acid chlorides R¹—W—Cl with for example potassium isocyanate or isothiocyanate respectively.

Compounds of formula (4) may be made from compounds of formula (2) (wherein R² and Y are as defined for a compound of formula (I)) as illustrated above in Scheme 1.

It will be appreciated that certain of the various ring substituents in the compounds of the present invention (for example substituents on R¹) may be introduced by standard aromatic substitution reactions or generated by conventional functional group modifications either prior to or immediately following the processes mentioned above, and as such are included in the process aspect of the invention. Such reactions may convert one compound of the formula (I) into another compound of the formula (I). Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogen group. Particular examples of modifications include the reduction of a nitro group to an amino group by for example, catalytic hydrogenation with a nickel catalyst or treatment with iron in the presence of hydrochloric acid with heating; oxidation of alkylthio to alkanesulfinyl or alkanesulfonyl.

If not commercially available, the necessary starting materials for the procedures such as those described above may be made by procedures which are selected from standard organic chemical techniques, techniques which are analogous to the synthesis of known, structurally similar compounds, techniques which are described or illustrated in the references given above, or techniques which are analogous to the above described procedure or the procedures described in the examples. The reader is further referred to Advanced Organic Chemistry, 5^{th} Edition, by Jerry March and Michael Smith, published by John Wiley & Sons 2001, for general guidance on reaction conditions and reagents.

It will be appreciated that some intermediates to compounds of the formula (I) are also novel and these are provided as separate independent aspects of the invention. In particular, compounds of formula (4) form a further aspect of the invention.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991).

Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

Examples of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl or SEM may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The protecting groups may be removed at any convenient stage in the synthesis using conventional techniques well known in the chemical art, or they may be removed during a later reaction step or work-up.

The skilled organic chemist will be able to use and adapt the information contained and referenced within the above references, and accompanying Examples therein and also the examples herein, to obtain necessary starting materials, and products.

The removal of any protecting groups and the formation of a salt are within the skill of an ordinary organic chemist using standard techniques. Furthermore, details on the these steps has been provided hereinbefore.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

In a further aspect of the invention, there is provided a compound of formula (I), (IA), (IB) and/or (IC) obtainable by a process as described hereinbefore or as shown in the Examples.

According to a further aspect of the invention there is provided a pharmaceutical composition which comprises a compound of formula (I), (IA), (IB) and/or (IC) as defined hereinbefore or a pharmaceutically-acceptable salt thereof, in association with a pharmaceutically-acceptable excipient or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing). In general, compositions in a form suitable for oral use are preferred.

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an allylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

According to a further aspect of the present invention there is provided a compound of formula (I), (IA), (IB) and/or (IC) or a pharmaceutically acceptable salt thereof as defined hereinbefore for use in a method of treatment of the human or animal body by therapy.

We have found that compounds of the present invention inhibit DGAT1 activity and are therefore of interest for their blood glucose-lowering effects.

A further feature of the present invention is a compound of formula (I), (IA), (IB) and/or (IC) or a pharmaceutically-acceptable salt thereof for use as a medicament.

Conveniently this is a compound of formula (I), (IA), (IB) and/or (IC), or a pharmaceutically-acceptable salt thereof, for use as a medicament for producing an inhibition of DGAT1 activity in a warm-blooded animal such as a human being.

Particularly this is a compound of formula (I), (IA), (IB) and/or (IC), or a pharmaceutically-acceptable salt thereof, for use as a medicament for treating diabetes mellitus and/or obesity in a warm-blooded animal such as a human being.

Thus according to a further aspect of the invention there is provided the use of a compound of formula (I), (IA), (IB) and/or (IC), or a pharmaceutically-acceptable salt thereof in the manufacture of a medicament for use in the production of an inhibition of DGAT1 activity in a warm-blooded animal such as a human being.

Thus according to a further aspect of the invention there is provided the use of a compound of formula (I), (IA), (IB) and/or (IC), or a pharmaceutically-acceptable salt thereof in the manufacture of a medicament for use in the treatment of diabetes mellitus and/or obesity in a warm-blooded animal such as a human being.

According to a further aspect of the invention there is provided a pharmaceutical composition which comprises a compound of formula (I), (IA), (IB) and/or (IC) as defined hereinbefore or a pharmaceutically-acceptable salt thereof, in association with a pharmaceutically-acceptable excipient or carrier for use in producing an inhibition of DGAT1 activity in an warm-blooded animal, such as a human being.

According to a further aspect of the invention there is provided a pharmaceutical composition which comprises a compound of formula (I), (IA), (IB) and/or (IC) as defined hereinbefore or a pharmaceutically-acceptable salt thereof, in association with a pharmaceutically-acceptable excipient or carrier for use in the treatment of diabetes mellitus and/or obesity in an warm-blooded animal, such as a human being.

According to a further feature of the invention there is provided a method for producing an inhibition of DGAT1 activity in a warm-blooded animal, such as a human being, in need of such treatment which comprises administering to said animal an effective amount of a compound of formula (I), (IA), (IB) and/or (IC) or a pharmaceutically-acceptable salt thereof as defined hereinbefore.

According to a further feature of the invention there is provided a method of treating diabetes mellitus and/or obesity in a warm-blooded animal, such as a human being, in need of such treatment which comprises administering to said animal an effective amount of a compound of formula (I), (IA), (IB) and/or (IC) or a pharmaceutically-acceptable salt thereof as defined hereinbefore.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

As stated above compounds defined in the present invention are of interest for their ability to inhibit the activity of DGAT1. A compound of the invention may therefore be useful for the prevention, delay or treatment of a range of disease states including diabetes mellitus, more specifically type 2 diabetes mellitus (T2DM) and complications arising there from (for example retinopathy, neuropathy and nephropathy), impaired glucose tolerance (IGT), conditions of impaired fasting glucose, metabolic acidosis, ketosis, dysmetabolic syndrome, arthritis, osteoporosis, obesity and obesity related disorders, peripheral vascular disease, (including intermittent claudication), cardiac failure and certain cardiac myopathies, myocardial ischaemia, cerebral ischaemia and reperfusion, muscle weakness, hyperlipidaemias, Alzheimer's disease, atherosclerosis, infertility, polycystic ovary syndrome, various immunomodulatory diseases (such as psoriasis), HIV infection, inflammatory bowel syndrome, inflammatory bowel disease (such as Crohn's disease and ulcerative colitis.

In particular, the compounds of the present invention are of interest for the prevention, delay or treatment of diabetes mellitus and/or obesity and/or obesity related disorders. In one aspect, the compounds of the invention are used for prevention, delay or treatment of diabetes mellitus. In another aspect, the compounds of the invention are used for prevention, delay or treatment of obesity. In a further aspect, the compounds of the invention are used for prevention, delay or treatment of obesity related disorders.

The inhibition of DGAT1 activity described herein may be applied as a sole therapy or in combination with one or more other substances and/or treatments for the indication being treated. Such conjoint treatment may be achieved by way of the simultaneous, sequential or separate administration of the individual components of the treatment. Simultaneous treatment may be in a single tablet or in separate tablets. For example such conjoint treatment may be beneficial in the treatment of metabolic syndrome [defined as abdominal obesity (as measured by waist circumference against ethnic and gender specific cut-points) plus any two of the following: hypertriglyceridemia (>150 mg/dl; 1.7 mmol/l); low HDLc (<40 mg/dl or <1.03 mmol/l for men and <50 mg/dl or 1.29 mmol/l for women) or on treatment for low HDL (high density lipoprotein); hypertension (SBP≧130 mmHg DBP≧85 mmHg) or on treatment for hypertension; and hyperglycemia (fasting plasma glucose≧100 mg/dl or 5.6 mmol/l or impaired glucose tolerance or pre-existing diabetes mellitus)—International Diabetes Federation & input from IAS/NCEP].

Such conjoint treatments may include the following main categories:

1) Anti-obesity therapies such as those that cause weight loss by effects on food intake, nutrient absorption or energy expenditure, such as orlistat, sibutramine and the like.
2) Insulin secretagogues including sulphonylureas (for example glibenclamide, glipizide), prandial glucose regulators (for example repaglinide, nateglinide);
3) Agents that improve incretin action (for example dipeptidyl peptidase IV inhibitors, and GLP-1 agonists);
4) Insulin sensitising agents including PPARgamma agonists (for example pioglitazone and rosiglitazone), and agents with combined PPARalpha and gamma activity;
5) Agents that modulate hepatic glucose balance (for example metformin, fructose 1, 6 bisphosphatase inhibitors, glycogen phopsphorylase inhibitors, glycogen synthase kinase inhibitors, glucokinase activators);
6) Agents designed to reduce the absorption of glucose from the intestine (for example acarbose);
7) Agents that prevent the reabsorption of glucose by the kidney (SGLT inhibitors);
8) Agents designed to treat the complications of prolonged hyperglycaemia (for example aldose reductase inhibitors);
9) Anti-dyslipidaemia agents such as, HMG-CoA reductase inhibitors (eg statins); PPARα-agonists (fibrates, eg gemfibrozil); bile acid sequestrants (cholestyramine); cholesterol absorption inhibitors (plant stanols, synthetic inhibitors); bile acid absorption inhibitors (IBATi) and nicotinic acid and analogues (niacin and slow release formulations);
10) Antihypertensive agents such as, β-blockers (eg atenolol, inderal); ACE inhibitors (eg lisinopril); Calcium antagonists (eg. nifedipine); Angiotensin receptor antagonists (eg candesartan), α antagonists and diuretic agents (eg. furosemide, benzthiazide);
11) Haemostasis modulators such as, antithrombotics, activators of fibrinolysis and antiplatelet agents; thrombin antagonists; factor Xa inhibitors; factor VIIa inhibitors); antiplatelet agents (eg. aspirin, clopidogrel); anticoagulants (heparin and Low molecular weight analogues, hirudin) and warfarin;
12) Agents which antagonise the actions of glucagon; and
13) Anti-inflammatory agents, such as non-steroidal anti-inflammatory drugs (eg. aspirin) and steroidal anti-inflammatory agents (eg. cortisone).

In addition to their use in therapeutic medicine, compounds of formula (I), (IA), (IB) and/or (IC) and their pharmaceutically-acceptable salts are also useful as pharmacological tools in the development and standardisation of in vitro and in vivo test systems for the evaluation of the effects of inhibitors of DGAT1 activity in laboratory animals such as cats, dogs, rabbits, monkeys, rats and mice, as part of the search for new therapeutic agents.

As indicated above, all of the compounds, and their corresponding pharmaceutically-acceptable salts, are useful in inhibiting DGAT1. The ability of the compounds of formula (I), (IA), (IB) and/or (IC) and their corresponding pharmaceutically-acceptable acid addition salts, to inhibit DGAT1 may be demonstrated employing the following enzyme assay:

### Human Enzyme Assay

The in vitro assay to identify DGAT1 inhibitors uses human DGAT1 expressed in insect cell membranes as the enzyme source (Proc. Natl. Acad. Sci. 1998, 95, 13018-13023). Briefly, sf9 cells were infected with recombinant baculovirus containing human DGAT1 coding sequences and harvested after 48 h. Cells were lysed by sonication and membranes isolated by centrifuging at 28000 rpm for 1 h at 4° C. on a 41% sucrose gradient. The membrane fraction at the interphase was collected, washed, and stored in liquid nitrogen.

DGAT1 activity was assayed by a modification of the method described by Coleman (Methods in Enzymology 1992, 209, 98-102). Compound at 1-10 μM was incubated with 0.4 μg membrane protein, 5 mM MgCl₂, and 100 μM 1,2dioleoyl-sn-glycerol in a total assay volume of 200 μl in plastic tubes. The reaction was started by adding ¹⁴C oleoyl coenzyme A (30 μM final concentration) and incubated at room temperature for 30 minutes. The reaction was stopped by adding 1.5 mL 2-propanol:heptane:water (80:20:2). Radioactive triolein product was separated into the organic phase by adding 1 mL heptane and 0.5 mL 0.1 M carbonate buffer pH 9.5. DGAT1 activity was quantified by counting aliquots of the upper heptane layer by liquid scintillography.

Using this assay the compounds generally show activity with IC₅₀<10 μM, preferably <1 μM. Example 26 showed an IC₅₀=0.33 μM

The ability of the compounds of formula (I), and their corresponding pharmaceutically-acceptable acid salts, to inhibit DGAT1 may further be demonstrated employing the following whole cell assays 1) and 2):

### 1) Measurement of Triglyceride Synthesis in 3T3 Cells

Mouse adipocyte 3T3 cells were cultured to confluency in 6 well plates in new born calf serum containing media. Differentiation of the cells was induced by incubating in medium containing 10% foetal calf serum, 1 μg/mL insulin, 0.25 μM dexamethasone and 0.5 mM isobutylmethyl xanthine. After 48 h the cells were maintained in medium containing 10% foetal calf serum and 1 μg/mL insulin for a further 4-6 days. For the experiment, the medium was changed to serum-free medium and the cells pre-incubated with compound solubilised in DMSO (final concentration 0.1%) for 30 minutes. De novo lipogenesis was measured by the addition of 0.25 mM sodium acetate plus 1 μCi/mL ¹⁴C-sodium acetate to each well for a further 2 h (J. Biol. Chem., 1976, 251, 6462-6464). The cells were washed in phosphate buffered saline and solubilised in 1% sodium dodecyl sulfate. An aliquot was removed for protein determination using a protein estimation kit (Perbio) based on the method of Lowry (J. Biol. Chem., 1951, 193, 265-275). The lipids were extracted into the organic phase using a heptane:propan-2-ol:water (80:20:2) mixture followed by aliquots of water and heptane according to the method of Coleman (Methods in Enzymology, 1992, 209, 98-104). The organic phase was collected and the solvent evaporated under a stream of nitrogen. The extracts solubilised in iso-hexane:acetic acid (99:1) and lipids separated via normal phase high performance liquid chromatography (HPLC) using a Lichrospher diol-5, 4×250 mm column and a gradient solvent system of iso-hexane:acetic acid (99:1) and iso-hexane:propan-2-ol:acetic acid (85:15:1), flow rate of 1 mL/minute according to the method of Silversand and Haux (1997). Incorporation of radiolabel into the triglyceride fraction was analysed using a Radiomatic Flo-one Detector (Packard) connected to the HPLC machine.

### 2) Measurement of Triglyceride Synthesis in MCF7 Cells

Human mammary epithelial (MCF7) cells were cultured to confluency in 6 well plates in foetal calf serum containing media. For the experiment, the medium was changed to serum-free medium and the cells pre-incubated with compound solubilised in DMSO (final concentration 0.1%) for 30 minutes. De novo lipogenesis was measured by the addition of 50 μM sodium acetate plus 3 μCi/mL ¹⁴C-sodium acetate to each well for a further 3 h (J. Biol. Chem., 1976, 251, 6462-6464). The cells were washed in phosphate buffered saline and solubilised in 1% sodium dodecyl sulfate. An aliquot was removed for protein determination using a protein estimation kit (Perbio) based on the method of Lowry (J. Biol. Chem., 1951, 193, 265-275). The lipids were extracted into the organic phase using a heptane:propan-2-ol:water (80:20:2) mixture followed by aliquots of water and heptane according to the method of Coleman (Methods in Enzymology, 1992, 209, 98-104). The organic phase was collected and the solvent evaporated under a stream of nitrogen. The extracts solubilised in iso-hexane:acetic acid (99:1) and lipids separated via normal phase high performance liquid chromatography (HPLC) using a Lichrospher diol-5, 4×250 mm column and a gradient solvent system of iso-hexane:acetic acid (99:1) and iso-hexane:propan-2-ol:acetic acid (85:15:1), flow rate of 1 mL/minute according to the method of Silversand and Haux (J. Chromat. B, 1997, 703, 7-14). Incorporation of radiolabel into the triglyceride fraction was analysed using a Radiomatic Flo-one Detector (Packard) connected to the HPLC machine.

In the above other pharmaceutical composition, process, method, use and medicament manufacture features, the alternative and preferred embodiments of the compounds of the invention described herein also apply.

### EXAMPLES

The invention will now be illustrated by the following Examples in which, unless stated otherwise:

(i) temperatures are given in degrees Celsius (° C.); operations were carried out at room or ambient temperature, that is, at a temperature in the range of 18-25° C. and under an atmosphere of an inert gas such as argon;
(ii) organic solutions were dried over anhydrous magnesium sulfate; evaporation of solvent was carried out using a rotary evaporator under reduced pressure (600-4000 Pa; 4.5-30 mmHg) with a bath temperature of up to 60° C.;
(iii) chromatography means flash chromatography on silica gel; where a Biotage cartridge is referred to this means a cartridge containing KP-SIL™ silica, 60 Å, particle size 32-63 mM, supplied by Biotage, a division of Dyax Corp., 1500 Avon Street Extended, Charlottesville, Va. 22902, USA;
(iv) in general, the course of reactions was followed by TLC and reaction times are given for illustration only;
(v) yields are given for illustration only and are not necessarily those which can be obtained by diligent process development; preparations were repeated if more material was required;
(vi) where given, NMR data (¹H) is in the form of delta values for major diagnostic protons, given in parts per million (ppm) relative to tetramethylsilane (TMS), determined at 300 or 400 MHz (unless otherwise stated) using perdeuterio dimethyl sulfoxide (DMSO-d₆) as solvent, unless otherwise stated; peak multiplicities are shown thus: s, singlet; d, doublet; dd, doublet of doublets; dt, doublet of triplets; dm, doublet of multiplets; t, triplet, q, quartet; m, multiplet; br, broad;
(vii) chemical symbols have their usual meanings; SI units and symbols are used;
(viii) solvent ratios are given in volume:volume (v/v) terms;
(ix) mass spectra (MS) (loop) were recorded on a Micromass Platform LC equipped with HP 1100 detector; unless otherwise stated the mass ion quoted is (MH⁺);
(x) LCMS (liquid chromatography-mass spectrometry) were recorded on a system comprising Waters 2790 LC equipped with a Waters 996 Photodiode array detector and Micromass ZMD MS, using a Phenomenex® Gemini 5u C18 110A 50×2 mm column and eluting with a flow rate of 1.1 ml/min with 5% (Water/Acetonitrile (1:1)+1% formic acid) and a gradient increasing from 0-95% of acetonitrile over the first 4 minutes, the balance (95-0%) being water and where HPLC Retention Times are reported these are in minutes in this system unless otherwise stated; unless otherwise stated the mass ion quoted is (MH⁺);
(xi) where phase separation cartridges are stated then ISOLUTE Phase Separator 70 ml columns, supplied by Argonaut Technologies, New Road, Hengoed, Mid Glamorgan, CF82 8AU, United Kingdom, were used;
(xii) where a SiliCycle cartridge is referred to this means a cartridge containing Ultra Pure Silica Gel particle size 230-400 mesh, 40-63 um pore size, supplied by SiliCycle Chemical Division, 1200 Ave St-Jean-Baptiste, Suite 114, Quebec City, Quebec, G2E 5E8, CANADA;
(xiii) where an Isco Companion is referred to then a Combiflash companion chromatography instrument, supplied by ISOC Inc. Address Teledyne ISOC Inc, 4700 Superior Street, Lincoln, Nebr. 68504, USA, was used;
(xiv) where a microwave is referred to this means a Biotage Initiator sixty or Smith Creator microwave, supplied by Biotage, a division of Dyax Corp., 1500 Avon Street Extended, Charlottesville, Va. 22902, USA;
(xv) where a centrifuge is referred to this means a Genevac EZ-2plus, supplied by Genevac Limited, The Sovereign Centre, Farthing Road, Ipswich, IP1 5AP, UK;
(xvi) Reverse phase preparative HPLC separations were run on standard Gilson™ HPLC equipment using a 150×21.2 mm Phenomenex Luna 10 micron C18(2) 100A column, and a standard gradient elution method (5-95% acetonitrile gradient with water as co-solvent and 0.2% trifluoroacetic acid as modifier, 12.5 min gradient with a 2.5 min hold at 95% acetonitrile) run on Unipoint software.
(xvi) The following abbreviations may be used below or in the process section hereinbefore:
Et₂O diethyl ether
DMF dimethylformamide
DCM dichloromethane
MeOH methanol
EtOH ethanol
H₂O water
THF tetrahydrofuran
DMSO dimethylsulfoxide
EtOAc ethyl acetate
PS-CDI polymer supported carbonyldiimidazole
HCl hydrochloric acid
   All compound names were derived using the ACD NAME computer package or similar.

### Example 1

### 5-[(4-Chlorobenzoyl)amino]-N-(3-fluoro-4-morpholin-4-ylphenyl)-1,3,4-oxadiazole-2-carboxamide

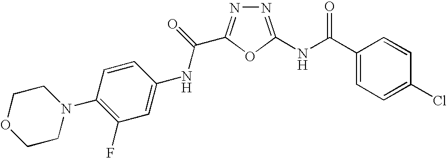

4-Chlorobenzoyl isothiocyanate (0.12 g, 0.60 mmol) was added to a stirred suspension of N-(3-fluoro-4-morpholin-4-ylphenyl)-2-hydrazino-2-oxoacetamide (Intermediate 5, 0.13 g, 0.50 mmol) in DMF (8 mL) and stirred at 50° C. for 2 hours. PS-CDI (0.85 g, 1.10 mmol) was added and the reaction was heated at 80° C. for a further 4 hours. The reaction was filtered and the resin washed with DMF (10 mL). The combined DMF solutions were concentrated in vacuo and the residue triturated with Et₂O to give the title compound as a yellow solid (62 mg, 29%): ¹H NMR: 12.60 (1H, s), 11.31 (1H, s), 8.05 (2H, d), 7.74-7.52 (4H, m), 7.13-7.01 (1H, m), 3.83-3.68 (4H, m), 3.06-2.92 (4H, m); MS MH⁺ 446.

### Examples 2 and 3

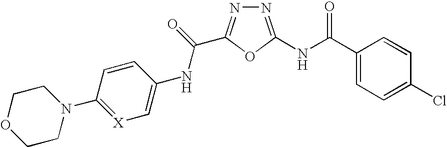

The following examples were prepared by the general procedure of Example 1 using 2-hydrazino-N-(4-morpholin-4-ylphenyl)-2-oxoacetamide (Intermediate 4) or 2-hydrazino-N-(6-morpholin-4-ylpyridin-3-yl)-2-oxoacetamide (Intermediate 6).

| Exam- | | | MS |
|---|---|---|---|
| ple | X | ¹H NMR | MH⁺ |
| 2 | CH | 10.70 (1H, s), 8.08 (2H, d), 7.67 (2H, d), 7.47 | 428 |
| | | (2H, d), 6.94 (2H, d), 3.83-3.67 (4H, m), 3.14- | |
| | | 3.00 (4H, m) | |
| 3 | N | 10.85 (1H, s), 8.54 (1H, s), 8.02 (2H, d), 7.83 | 429 |
| | | (2H, d), 7.5 (2H, d), 6.86 (1H, d), 3.71-3.6(4H, m), | |
| | | 3.45-3.37(4H, m) | |

### Example 4

### Methyl (trans-4-{4-[({5-[(4-chlorobenzoyl)amino]-1,3,4-oxadiazol-2-yl}carbonyl)amino]phenyl}cyclohexyl)acetate

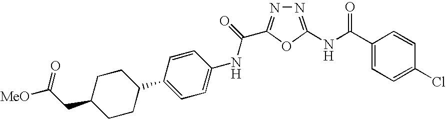

The title compound was prepared by the general procedure of Example 1, using methyl [trans-4-(4-{[hydrazino(oxo)acetyl]amino}phenyl)cyclohexyl]acetate (Intermediate 12):

¹H NMR: 10.67 (1H, br.s), 8.05 (2H, d), 7.69 (2H, d), 7.45 (2H, d), 7.20 (2H, d), 7.10 (1H, br.s), 3.60 (3H, s), 2.24 (2H, d), 1.84-1.68 (6H, m), 1.44 (2H, m), 1.12 (2H, m);

MS MH⁺ 497.

### Example 5

### (trans-4-{4-[({5-[(4-Chlorobenzoyl)amino]-1,3,4-oxadiazol-2-yl}carbonyl)amino]phenyl}cyclohexyl)acetic acid

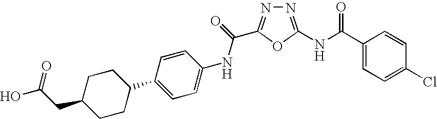

Lithium hydroxide (2 mg, 47.6 μmol) was added to a solution of methyl (trans-4-{4-[({5-[(4-chlorobenzoyl)amino]-1,3,4-oxadiazol-2-yl}carbonyl)amino]phenyl}cyclohexyl)acetate (Example 4, 12 mg, 24.2 μmol) in MeOH/H₂O (1:1) (1 mL). The reaction was stirred at room temperature for 18 hours. The reaction was cooled in an ice bath and acidified to pH 5 with 2M HCl. The resulting precipitate was filtered off and dried under high vacuum to give the title compound as a white solid (9 mg, 75%): ¹H NMR: 12.48 (1H, br.s), 11.95 (1H, br.s), 11.09 (1H, s), 8.04 (2H, d), 7.70 (2H, d), 7.65 (2H, d), 7.25 (2H, d), 2.12 (2H, d), 1.87-1.66 (6H, m), 1.45 (2H, m), 1.10 (2H, m); MS MH⁺ 483.

### Example 6

### 5-(Benzoylamino)-N-(4-morpholin-4-ylphenyl)-1,3,4-oxadiazole-2-carboxamide

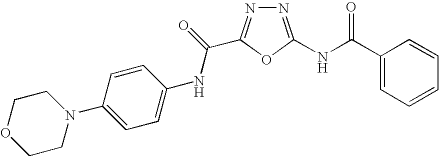

Benzoyl chloride (35 μl, 0.3 mmol) was added to a stirred suspension of potassium thiocyanate (29 mg, 0.3 mmol) in THF (5 mL) and allowed to stir at room temperature for 2 hours. The suspension was then added to 2-hydrazino-N-(4-morpholin-4-ylphenyl)-2-oxoacetamide (Intermediate 4, 66 mg, 0.25 mmol) in DMF (5 mL) and stirred at 50° C. for a further 2 hours. PS-CDI (384 mg, 0.5 mmol) was then added and the reaction was heated to 80° C. for 16 hours. The resin was removed by filtration and washed with DMF (10 mL), and then the combined filtrates were concentrated in vacuo. The residue was triturated with Et₂O to give the title compound as a fawn solid (30 mg, 30%): ¹H NMR: 12.45 (1H, s), 11.01 (1H, s), 8.05 (2H, d), 7.73-7.64 (3H, m), 7.62-7.54 (2H, m), 6.96 (2H, d), 3.79-3.71 (4H, m), 3.14-3.05 (4H, m); MS MH⁺ 394.

### Examples 7-17

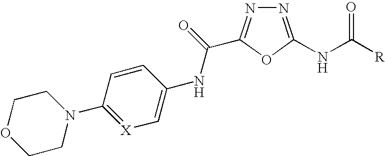

The following examples were prepared by the general procedure of Example 6, using commercially available acid chlorides and either 2-hydrazino-N-(4-morpholin-4-ylphenyl)-2-oxoacetamide (Intermediate 4) or 2-hydrazino-N-(6-morpholin-4-ylpyridin-3-yl)-2-oxoacetamide (Intermediate 6).

| | | | | MS |
|---|---|---|---|---|
| Example | X | R | ¹H NMR | MH⁺ |
| 7 | CH | 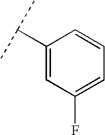 | 10.97 (2H, s), 7.93-7.78 (2H, m), 7.67 (2H, d), 7.64-7.57 (1H, m), 7.53-7.45 (1H, m), 6.95 (2H, d), 3.80-3.69 (4H, m), 3.15-3.05 (4H, m) | 412 |
| 8 | CH | 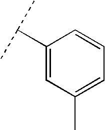 | 12.40 (1H, s), 10.96 (1H, s), 7.91-7.80 (2H, m), 7.68 (2H, d), 7.50-7.38 (2H, m), 6.95 (2H, d), 3.81-3.69 (4H, m), 3.13-3.04 (4H, m), 2.43 (3H, s) | 408 |
| 9 | CH | 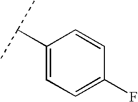 | 10.66 (1H, s), 8.16-8.08 (2H, m), 8.01-7.91 (1H, m), 7.67 (2H, d), 7.32- 7.23 (2H, m), 6.94 (2H, d), 3.79-3.70 (4H, m), 3.12-3.04 (4H, m) | 412 |
| 10 | CH | 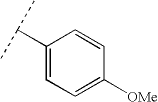 | 12.25 (1H, s), 10.89 (1H, s), 8.04 (2H, d), 7.67 (2H, d), 7.10 (2H, d), 6.96 (2H, d), 3.89 (3H, s), 3.77-3.72 (4H, m), 3.14-3.05 (4H, m) | 424 |
| 11 | CH | 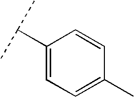 | 10.98 (1H, s), 10.36 (1H, s), 7.95 (2H, d), 7.70-7.64 (2H, m), 7.38 (2H, d), 7.00-6.89 (2H, m), 3.80-3.70 (4H, m) 3.14-3.03 (4H, m), 2.44 (3H, s) | 408 |
| 12 | CH | 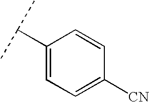 | 10.52 (1H, s), 8.21 (3H, d), 7.82 (2H, d), 7.66 (2H, d), 6.94 (2H, d), 3.78- 3.69 (4H, m), 3.12-3.04 (4H, m) | 419 |
| 13 | CH | 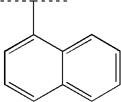 | 12.6 (1H, s), 10.99 (1H, s), 8.3 (1H, d), 8.16 (1H, d), 8.06 (1H, m), 7.91 (1H, d), 7.71-7.60 (5H, m), 6.95 (2H, d), 3.78-3.69 (4H, m), 3.12-3.03 (4H, m) | 444 |
| 14 | CH | 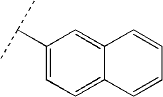 | 10.51 (1H, s), 8.64 (1H, s), 8.18 (1H, d), 8.04 (1H, m), 7.97-7.87 (2H, m), 7.68 (2H, d), 7.58-7.48 (2H, m), 6.94 (2H, d), 3.79-3.68 (4H, m), 3.1-3.0 (4H, m) | 444 |
| 15 | CH | 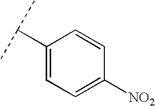 | 10.49 (1H, s), 8.27 (2H, d), 8.21 (2H, d), 7.65 (2H, d), 6.92 (2H, d), 3.8- 3.69 (4H, m), 3.12-3.02 (4H, m) | 438 |
| 16 | CH | 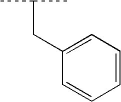 | 12.19 (1H, s), 10.9 (1H, s), 7.62 (2H, d), 7.40-7.20 (5H, m), 6.91 (2H, d), 3.81 (2H, s), 3.75-3.66 (4H, m), 3.14-3.01 (4H, m) | 408 |
| 17 | N | 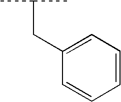 | 12.22 (1H, s), 11.09 (1H, s), 8.50 (1H, s), 7.99 (2H, d), 7.39-7.19 (5H, m), 6.95 (2H, d), 3.79 (2H, s), 3.77- 3.62 (4H, m), 3.49-3.38 (4H, m) | 409 |

### Examples 18-23

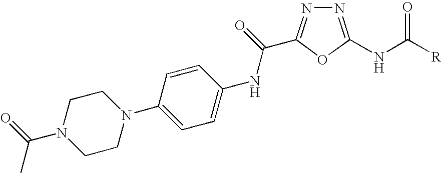

The following examples were prepared by the general procedure of Example 6, using commercially available acid chlorides and N-[4-(4-acetylpiperazin-1-yl)phenyl]-2-hydrazino-2-oxoacetamide (Intermediate 8).

| | | | MS |
|---|---|---|---|
| Example | R | ¹H NMR | MH⁺ |
| 18 | 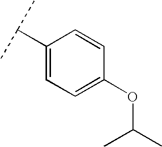 | 12.16 (1H, br.s), 10.93 (1H, s), 8.01 (2H, dd), 7.68 (2H, d), 7.06 (2H, d), 6.98 (2H, d), 4.77 (1H, septet), 3.61- 3.56 (4H, m), 3.12 (4H, dt), 2.05 (3H, s), 1.31 (6H, d) | 493 |
| 19 | 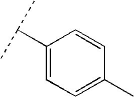 | 12.29 (1H, br.s), 10.95 (1H, s), 7.95 (2H, d), 7.68 (2H, d), 7.38 (2H, d), 6.98 (2H, d), 3.62-3.55 (4H, m), 3.12 (4H, dt), 2.41 (3H, s), 2.05 (3H, s) | 449 |
| 20 | 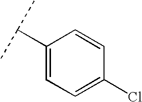 | 12.54 (1H, br.s), 10.80 (1H, s), 8.06 (2H, dt), 7.68 (2H, d), 7.56 (2H, dd), 6.97 (2H, d), 3.62-3.54 (4H, m), 3.11 (4H, dt), 2.04 (3H, d) | 469 |
| 21 | 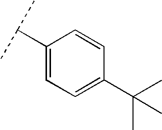 | 12.31 (1H, br.s), 10.94 (1H, s), 8.00 (2H, d), 7.68 (2H, d), 7.58 (2H, d), 6.98 (2H, d), 3.62-3.55 (4H, m), 3.12 (4H, dt), 2.05 (3H, s), 1.33 (9H, s) | 491 |
| 22 | 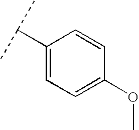 | 12.18 (1H, br.s), 10.98 (1H, s), 8.05 (2H, d), 7.68 (2H, d), 7.11 (2H, d), 7.00 (2H, d), 3.87 (3H, s), 3.63-3.56 (4H, m), 3.13 (4H, dt), 2.05 (3H, s) | 465 |
| 23 | 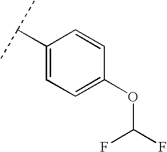 | 12.40 (1H, br.s), 10.99 (1H, s), 8.12 (2H, dt), 7.69 (2H, d), 7.42 (1H, t), 7.36 (2H, d), 7.00 (2H, d), 3.63-3.55 (4H, m), 3.13 (4H, dt), 2.05 (3H, s) | 501 |

### Example 24

### 5-{[2-(4-Chlorophenyl)-2-methylpropanoyl]amino}-N-(4-morpholin-4-ylphenyl)-1,3,4-oxadiazole-2-carboxamide

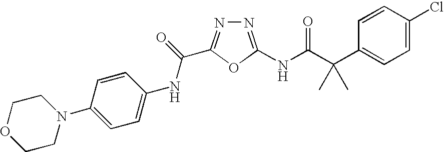

To a stirred solution of 2-(4-chlorophenyl)-2-methylpropionic acid (99 mg, 0.5 mmol) in THF (5 mL) was added 1-chloro-N,N,2-trimethylprop-1-en-1-amine (70 μt, 0.5 mmol) and stirring was continued for 1 hour. Potassium thiocyanate (50 mg, 0.515 mmol) was added and stirring continued for 1 hour. 2-Hydrazino-N-(4-morpholin-4-ylphenyl)-2-oxoacetamide (Intermediate 4, 132 mg, 0.5 mmol,) was added with DMF (5 mL) and the reaction was heated to 60° C. for 2 hours. PS-CDI (900 mg, 1.1 mmol) was added and the reaction was heated at 80° C. for a further 2 hours. The resin was removed via filtration and washed with DMF (10 mL) and the combined filtrates were evaporated to dryness. The resulting gum was portioned between ethyl acetate/water (100 mL) and the organic layer was removed, dried and concentrated in vacuo. The resulting foam was purified by chromatography on silica gel eluting with MeOH/DCM (0-10%) to give the title compound as pale yellow solid (105 mg, 45%):

¹H NMR: 11.47 (1H, s), 10.95 (1H, s), 7.64 (2H, d), 7.45 (2H, d), 7.36 (2H, d), 6.94 (2H, d), 3.80-3.68 (4H, m), 3.14-3.04 (4H, m), 1.62 (6H, s); MS MH⁺ 470.

### Examples 25-32

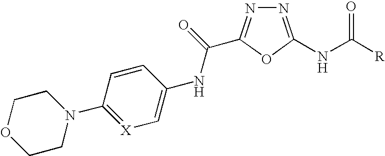

The following examples were prepared by the general procedure of Example 24, using commercially available carboxylic acids and either 2-hydrazino-N-(4-morpholin-4-ylphenyl)-2-oxoacetamide (Intermediate 4) or 2-hydrazino-N-(6-morpholin-4-ylpyridin-3-yl)-2-oxoacetamide (Intermediate 6).

| | | | | MS |
|---|---|---|---|---|
| Example | X | R | ¹H NMR | MH⁺ |
| 25 | CH | 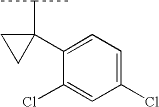 | 11.02 (1H, s), 10.95 (1H, s), 7.69- 7.60 (3H, m), 7.54 (1H, d), 7.45 (1H, d), 6.95 (2H, d), 3.80-3.69 (4H, m), 3.13-3.03 (4H, m), 1.78 (2H, s), 1.33 (2H, s) | 501 |
| 26 | CH | 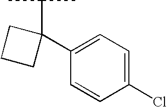 | 11.79 (1H, s), 10.92 (1H, s), 7.64 (2H, d), 7.51 (4H, s), 6.95 (2H, d), 3.79-3.68 (4H, m), 3.15-3.02 (4H, m), 2.93-2.78 (4H, m), 1.96-1.74 (2H, m) | 482 |
| 27 | CH | 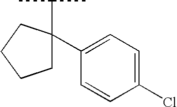 | 11.54 (1H, s), 10.93 (1H, s), 7.63 (2H, d), 7.49-7.37 (4H, m), 6.94 (2H, d), 3.82-3.68 (4H, m), 3.14-3.02 (4H, m), 2.66-2.56 (2H, m), 2.03- 1.90 (2H, m), 1.75-1.59 (4H, m) | 496 |
| 28 | CH | 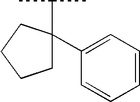 | 11.50 (1H, s), 10.92 (1H, s), 7.64 (2H, d), 7.43-7.34 (4H, m), 7.32-7.25 (1H, m), 6.95 (2H, d), 3.80-3.69 (4H, m), 3.13-3.04 (4H, m), 2.66-2.57 (2H, m), 2.03-1.92 (2H, m) | 463 |
| 29 | N | 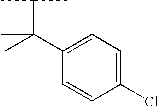 | 11.54 (1H, s), 11.00 (1H, s), 8.54 (1H, s), 7.93 (1H, d), 7.46-7.33 (4H, m), 6.86 (1H, d), 3.77-3.64 (4H, m), 3.47-3.34 (4H, m), 1.66 (6H, s) | 471 |
| 30 | N | 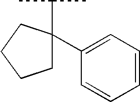 | 11.53 (1H, s), 11.09 (1H, s), 8.49 (1H, d), 7.44-7.34 (4H, m), 7.32-7.26 (1H, m), 6.93 (1H, d), 3.74-3.67 (4H, m), 3.47-3.40 (4H, m), 2.66-2.57 (2H, m), 2.05-1.92 (2H, m), 1.76- 1.58 (4H, m), | 464 |
| 31 | N | 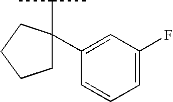 | 11.58 (1H, s), 11.06 (1H, s), 8.49 (1H, d), 7.97-7.91 (1H, m), 7.47-7.39 (1H, m), 7.25-7.18 (2H, m), 7.17- 7.09 (1H, m), 6.87 (1H, d), 3.75-3.67 (4H, m), 3.46-3.39 (4H, m), 2.70- 2.57 (2H, m), 2.03-1.93 (2H, m), 1.77-1.58 (4H, m) | 481 |
| 32 | N | 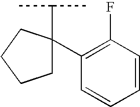 | 11.42 (1H, s), 11.06 (1H, s), 8.52 (1H, s), 7.94 (1H, d), 7.56-7.48 (1H, m), 7.42-7.32 (1H, m), 7.29-7.23 (1H, m), 7.21-7.12 (1H, m), 6.88 (1H d), 3.76-3.62 (4H, m), 3.55-3.23 (4H, m), 2.09-1.92 (2H, m), 1.79-1.61 (4H, m) | 481 |

### Examples 33-60

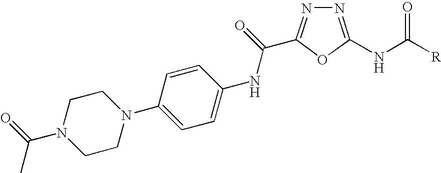

The following examples were prepared by the general procedure of Example 24, using commercially available carboxylic acids and N-[4-(4-acetylpiperazin-1-yl)phenyl]-2-hydrazino-2-oxoacetamide (Intermediate 8). The examples were purified by reverse phase preparative HPLC.

| | | | MS |
|---|---|---|---|
| Example | R | ¹H NMR | MH⁺ |
| 33 | 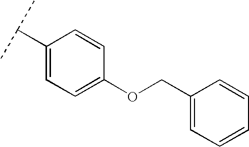 | 12.18 (1H, s), 10.97 (1H, s), 8.04 (2H, d), 7.68 (2H, d), 7.51- 7.33 (5H, m), 7.19 (2H, d), 6.98 (2H, d), 5.23 (2H, s), 3.61-3.55 (4H, m), 3.12 (4H, dt), 2.05 (3H, s) | 541 |
| 34 | 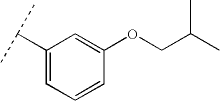 | 12.32 (1H, s), 10.98 (1H, s), 7.68 (2H, d), 7.63-7.58 (2H, m), 7.47 (1H, t), 7.24 (1H, dd), 6.99 (2H, d), 3.85 (2H, d), 3.62-3.55 (4H, m), 3.12 (4H, dt), 2.12-2.01 (4H, m), 1.02 (6H, d) | 507 |
| 35 | 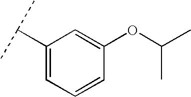 | 12.31 (1H, s), 10.98 (1H, s), 7.68 (2H, d), 7.61-7.55 (2H, m), 7.46 (1H, t), 7.21 (1H, dd), 6.99 (2H, d), 4.73 (1H, quintet), 3.62- 3.55 (4H, m), 3.12 (4H, dt), 2.05 (3H, s), 1.32 (6H, d) | |
| 36 | 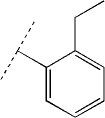 | 12.37 (1H, s), 10.97 (1H, s), 7.68 (2H, d), 7.57 (1H, d), 7.50 (1H, td), 7.41-7.32 (2H, m), 6.98 (2H, d), 3.62-3.55 (4H, m), 3.12 (4H, dt), 2.78 (2H, q), 2.05 (3H, s), 1.20 (3H, t) | 463 |
| 37 | 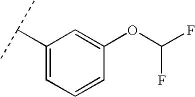 | 12.49 (1H, br.s), 10.99 (1H, s), 7.93 (1H, d), 7.83 (1H, s), 7.71- 7.62 (3H, m), 7.53-7.47 (1H, m), 7.33 (1H, t), 6.99 (2H, d), 3.62- 3.55 (4H, m), 3.12 (4H, dt), 2.05 (3H, s) | 501 |
| 38 | 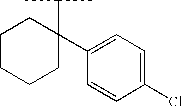 | 11.37 (1H, s), 10.91 (1H, s), 7.65 (2H, d), 7.49-7.40 (4H, m), 6.97 (2H, d), 3.62-3.54 (4H, m), 3.11 (4H, dt), 2.04 (3H, s), 1.82- 1.71 (2H, m), 1.68-1.44 (5H, m), 1.36-1.24 (1H, m) Note: 2H obscured by DMSO | 551 |
| 39 | 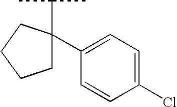 | 11.50 (1H, s), 10.90 (1H, s), 7.64 (2H, d), 7.47-7.37 (4H, m), 6.96 (2H, d), 3.61-3.54 (4H, m), 3.11 (4H, dt), 2.64-2.56 (2H, m), 2.05 (3H, s), 2.01-1.91 (2H, m), 1.74-1.61 (4H, m) | 537 |
| 40 | 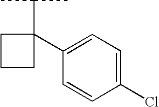 | 11.75 (1H, s), 10.90 (1H, s), 7.64 (2H, d), 7.47 (4H, s), 6.96 (2H, d), 3.61-3.54 (4H, m), 3.11 (4H, dt), 2.89-2.80 (2H, m), 2.04 (3H, s), 1.92-1.79 (2H, m) Note: 2H obscured by DMSO | 523 |
| 41 | 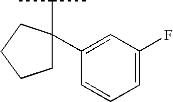 | 11.52 (1H, s), 10.90 (1H, s), 7.64 (2H, d), 7.47-7.40 (1H, m), 7.25-7.20 (2H, m), 7.16-7.10 (1H, m), 6.97 (2H, d), 3.61-3.55 (4H, m), 3.11 (4H, dt), 2.66-2.58 (2H, m), 2.04 (3H, s), 2.03-1.93 (2H, m), 1.76-1.61 (4H, m) | 521 |
| 42 | 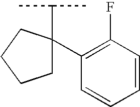 | 11.36 (1H, s), 10.90 (1H, s), 7.64 (2H, d), 7.52 (1H, td), 7.40- 7.34 (1H, m), 7.26 (1H, td), 7.21-7.14 (1H, m), 6.97 (2H, d), 3.61-3.55 (4H, m), 3.11 (4H, dt), 2.07-1.98 (5H, m), 1.75-1.67 (4H, m) Note: 2H obscured by DMSO | |
| 43 | 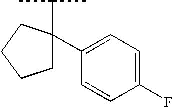 | 11.48 (1H, s), 10.90 (1H, s), 7.64 (2H, d), 7.43 (2H, dd), 7.21 (2H, t), 6.96 (2H, d), 3.61-3.54 (4H, m), 3.11 (4H, dt), 2.66-2.57 (2H, m), 2.04 (3H, s), 2.01-1.91 (2H, m), 1.75-1.60 (4H, m) | |
| 44 | 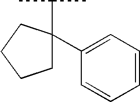 | 11.46 (1H, s), 10.90 (1H, s), 7.64 (2H, d), 7.43-7.35 (4H, m), 7.31-7.26 (1H, m), 6.96 (2H, d), 3.60-3.55 (4H, m), 3.11 (4H, dt), 2.66-2.57 (2H, m), 2.04 (3H, s), 2.03-1.93 (2H, m), 1.76-1.61 (4H, m) | |
| 45 | 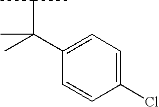 | 11.42 (1H, s), 10.91 (1H, s), 7.65 (2H, d), 7.48-7.43 (2H, m), 7.39-7.34 (2H, m), 6.97 (2H, d), 3.61-3.54 (4H, m), 3.11 (4H, dt), 2.04 (3H, s), 1.58 (6H, s) | |
| 46 | 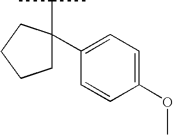 | 11.38 (1H, s), 10.90 (1H, s), 7.64 (2H, d), 7.34-7.29 (2H, m), 6.95 (4H, t), 3.75 (3H, s), 3.61- 3.55 (4H, m), 3.11 (4H, dt), 2.63-2.54 (2H, m), 2.04 (3H, s), 1.99-1.89 (2H, m), 1.74-1.59 (4H, m) | |
| 47 | 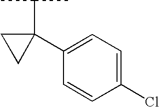 | 11.17 (1H, br.s), 10.91 (1H, s), 7.64 (2H, d), 7.45-7.39 (4H, m), 6.96 (2H, d), 3.61-3.54 (4H, m), 3.11 (4H, dt), 2.04 (3H, s), 1.57 (2H, dd), 1.23 (2H, dd) | 509 |
| 48 | 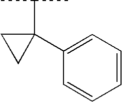 | 11.17 (1H, br.s), 10.92 (1H, s), 7.64 (2H, d), 7.44-7.28 (5H, m), 6.96 (2H, d), 3.61-3.54 (4H, m), 3.11 (4H, dt), 2.04 (3H, s), 1.55 (2H, dd), 1.22 (2H, dd) | |
| 49 | 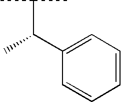 | 12.15 (1H, s), 10.89 (1H, s), 7.65 (2H, d), 7.40-7.25 (5H, m), 6.96 (2H, d), 3.96 (1H, q), 3.61- 3.54 (4H, m), 3.11 (4H, dt), 2.04 (3H, s), 1.44 (3H, d) | |
| 50 | 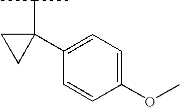 | 10.93 (1H, s), 10.92 (1H, s), 7.68-7.61 (2H, m), 7.35 (2H, d), 7.00-6.91 (4H, m), 3.77 (3H, s), 3.61-3.54 (4H, m), 3.11 (4H, dt), 2.04 (3H, s), 1.52 (2H, dd), 1.17 (2H, dd) | |
| 51 | 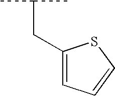 | 12.17 (1H, s), 10.82 (1H, s), 7.56 (2H, d), 7.36-7.33 (1H, m), 6.95-6.86 (4H, m), 3.95 (2H, s), 3.51-3.46 (4H, m), 3.08-2.97 (4H, m), 1.95 (3H, s) | 455 |
| 52 | 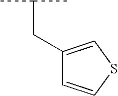 | 12.09 (1H, s), 10.81 (1H, s), 7.56 (2H, d), 7.44-7.40 (1H, m), 7.29 (1H, s), 7.00 (1H, d), 6.88 (2H, d), 3.73 (2H, s), 3.52-3.46 (4H, m), 3.08-2.97 (4H, m), 1.95 (3H, s) | 455 |
| 53 | 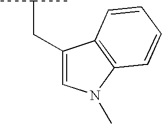 | 12.04 (1H, s), 10.80 (1H, s), 7.55 (2H, d), 7.50 (1H, d), 7.32 (1H, d), 7.19 (1H, s), 7.08 (1H, t), 6.96 (1H, t), 6.87 (2H, d), 3.79 (2H, s), 3.68 (3H, s), 3.51- 3.46 (4H, m), 3.08-2.96 (4H, m), 1.95 (3H, s) | 502 |
| 54 | 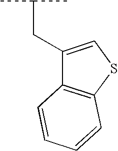 | 10.81 (1H, s), 10.45 (1H, s), 7.94-7.88 (1H, m), 7.84-7.76 (1H, m), 7.61-7.54 (3H, m), 7.37-7.27 (2H, m), 6.87 (2H, t), 4.01 (2H, s), 3.52-3.45 (4H, m), 3.07-2.96 (4H, m), 1.95 (3H, s) | 505 |
| 55 | 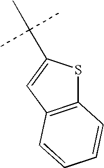 | 10.85 (2H, br s), 8.03-7.91 (2H, m), 7.59 (2H, d), 7.49-7.36 (3H, m), 6.92-6.85 (2H, m), 3.53-3.45 (4H, m), 3.09-2.97 (4H, m), 2.00 (3H, s) | 491 |
| 56 | 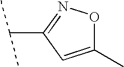 | 10.86 (2H, s), 7.57 (2H, d), 6.89 (2H, d), 6.66 (1H, s), 3.52-3.47 (4H, m), 3.24 (3H, s), 3.09-2.97 (4H, m), 1.96 (3H, s) | 440 |
| 57 | 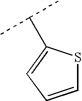 | 10.86 (2H, s), 8.06 (1H, br.s) 7.92 (1H, d), 7.58 (2H, d), 7.18 (1H, t), 6.89 (2H, d), 3.53-3.46 (4H, m), 3.03 (4H, dt), 1.96 (3H, s) | 441 |
| 58 | 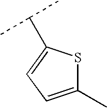 | 10.85 (2H, s), 7.88 (1H, br.s), 7.58 (2H, d), 6.89 (3H, d), 3.54- 3.45 (4H, m), 3.03 (4H, dd), 2.44 (3H, s), 1.96 (3H, s) | 455 |
| 59 | 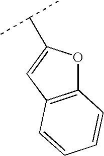 | 10.89 (2H, s), 7.93 (1H, br.s), 7.80 (1H, d), 7.67 (1H, d), 7.59 (2H, d), 7.47 (1H, s), 7.32 (1H, s), 6.90 (2H, d), 3.55-3.45 (4H, m), 3.04 (4H, dt), 1.96 (3H, s) | |
| 60 | 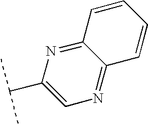 | 10.91 (2H, s), 9.46 (1H, s), 8.23- 8.13 (2H, m), 8.01-7.93 (2H, m), 7.66-7.56 (2H, m), 6.93-6.86 (2H, m), 3.54-3.46 (4H, m), 3.11-2.97 (4H, m), 1.96 (3H, s) | |

### Preparation of Starting Materials

### Intermediate 1: Methyl [(4-morpholin-4-yl phenyl)amino](oxo)acetate

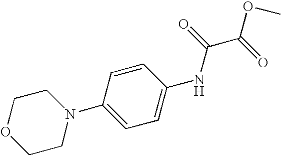

Methyl chloro(oxo)acetate (4.64 mL, 50 mmol) was added dropwise to an ice cooled solution of 4-morpholinoaniline (8.91 g, 50 mmol) and ethyldiisopropylamine (9.4 mL, 55 mmol) in DCM (125 mL). The reaction was stirred for 2 hours at room temperature then quenched with H₂O (100 mL). The organic layer was removed, dried, filtered and concentrated in vacuo to give the title compound (11.7 g, 89%): ¹HNMR: 10.63 (1H, s), 7.61 (2H, d), 6.92 (2H, d), 3.88 (3H, s), 3.78-3.68 (4H, m), 3.15-3.04 (4H, m); MS MH⁺ 265.

### Intermediates 2-3

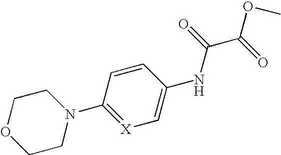

The following intermediates were prepared by the general procedure of Intermediate 1 using (3-fluoro-4-morpholin-4-ylphenyl)amine (J. Med. Chem. 1996, 39, 673-679) and a commercially available aniline. For Intermediate 3 pyridine was used as the base instead of ethyldiisopropylamine.

| | | | MS |
|---|---|---|---|
| Intermediate | X | ¹H NMR | MH⁺ |
| 2 | CF | 10.74 (1H, s), 7.65-6.97 (3H, m), 3.86-3.69 | 283 |
| | | (7H, m), 3.31-2.46 (4H, m) | |
| 3 | N | 10.74 (1H, s), 8.48 (1H, d), 7.91 (1H, dd), | 266 |
| | | 6.88 (1H, d), 3.85 (3H, s), 3.74-3.66 (4H, | |
| | | m), 3.45-3.37 (4H, m) | |

### Intermediate 4: 2-Hydrazino-N-(4-morpholin-4-ylphenyl)-2-oxoacetamide

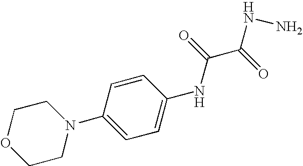

Hydrazine hydrate (1.25 mL, 25 mmol) was added to a stirred suspension of methyl [(4-morpholin-4-ylphenyl)amino](oxo)acetate (Intermediate 1, 6.6 g, 25 mmol) in MeOH (150 mL). The reaction was heated to 75° C. for 2 hours during which time the precipitate thickened. After cooling the precipitate was filtered and washed with Et₂O (50 mL) and dried to give the title compound (6.32 g, 94%): ¹H NMR: 10.38 (1H, s), 10.15 (1H, s), 7.69 (2H, d), 6.91 (2H, d), 3.75 (4H, m), 3.08 (4H, m); MS MH⁺ 265.

### Intermediates 5 and 6

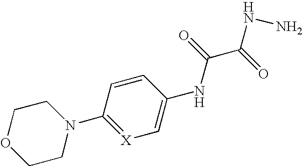

The following intermediates were prepared by the general procedure of Intermediate 4 using Intermediates 2 and 3.

| | | | MS |
|---|---|---|---|
| Intermediate | X | ¹H NMR | MH⁺ |
| 5 | CF | 7.67 (1H, dd), 7.60-7.54 (1H, m), 7.00 (1H, t), | 283 |
| | | 4.78-4.24 (3H, m), 3.71 (4H, t), 2.95 (4H, t) | |
| 6 | N | 10.54 (1H, s), 10.20 (1H, br.s), 8.54 (1H, s), | 266 |
| | | 7.95 (1H, d), 6.84 (1H, d), 4.60 (2H, br.s), | |
| | | 3.73-3.66 (4H, m), 3.44-3.36 (4H, m) | |

### Intermediate 7: Methyl {[4-(4-acetylpiperazin-1-yl)phenyl]amino}(oxo)acetate

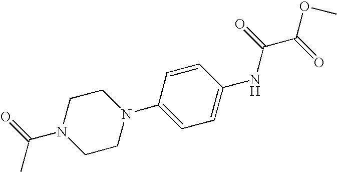

Intermediate 7 was prepared by the general procedure of Intermediate 1 using 4-(4-acetylpiperazin-1-yl)aniline. Triethylamine was used as the base instead of ethyldiisopropylamine: MS MH⁺ 306.

### Intermediate 8: N-[4-(4-acetylpiperazin-1-yl)phenyl]-2-hydrazino-2-oxoacetamide

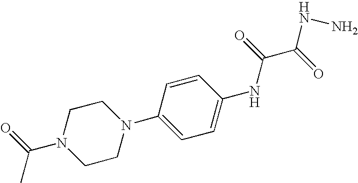

Intermediate 8 was prepared by the general procedure of Intermediate 4 using Intermediate 7:

MS MH⁺ 306.

### Intermediate 9: Methyl (trans-4-phenylcyclohexyl)acetate

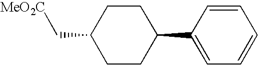

10% Pd/C (4.52 g) was added to a solution of methyl [trans-4-(4-{[(trifluoromethyl)sulfonyl]oxy}phenyl)cyclohexyl]acetate (prepared as described in Patent Application WO2004/047755) (8.10 g) in MeOH (150 mL). The resulting suspension was stirred for 16 hours under an atmosphere of hydrogen. The suspension was filtered through diatomaceous earth and concentrated in vacuo to give a slurry. This was extracted into EtOAc (300 mL). The organic extract was washed with saturated aqueous sodium hydrogen carbonate solution (75 mL) and then brine (75 mL). The organic layer was dried and concentrated in vacuo to give the title compound as an oil (4.72 g): ¹H NMR: 7.28-7.11 (5H, m), 3.58 (3H, s), 2.43 (1H+DMSO, m), 2.22 (2H, d), 1.83-1.67 (5H, m), 1.44 (2H, m), 1.13 (2H, m); MS MH⁺ 233.

### Intermediate 10: Methyl [trans-4-(4-aminophenyl)cyclohexyl]acetate

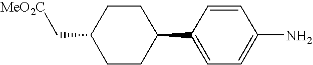

A mixture of 65% nitric acid (3.95 mL) and 95% sulphuric acid (4.97 mL) was added dropwise to a stirred solution of methyl (trans-4-phenylcyclohexyl)acetate (Intermediate 7; 4.71 g) in carbon tetrachloride (20 mL) at 5° C. and the solution was allowed to warm to ambient temperature and stirred for 16 hours. Ice/water (50 mL) was added and the mixture was extracted with DCM (2×40 mL). The organic extracts were combined, washed with brine (50 mL), dried, and concentrated in vacuo to give an oil. The oil was purified by flash chromatography on a 80 g Biotage™ silica column, using a gradient of 0-20% EtOAc in hexane as eluent to give crude methyl [trans-4-(4-nitrophenyl)cyclohexyl]acetate which was dissolved in EtOAc (30 mL). 10% Pd/C (0.40 g) was added and the resulting suspension was stirred at ambient temperature for 16 hours under an atmosphere of hydrogen. The suspension was filtered through diatomaceous earth and concentrated in vacuo to give a solid. This was purified by flash chromatography on a 40 g Biotage™ silica column using a gradient of 20-45% EtOAc in hexane as eluent to give the title compound as a solid (1.74 g):

¹H NMR: 6.83 (2H, d), 6.46 (2H, d), 4.72 (2H, s), 3.59 (3H, s), 2.23 (3H, m), 1.72 (5H, m), 1.35 (2H, m), 1.09 (2H, m); MS: MH⁺ 248.

### Intermediate 11: Methyl({4-[trans-4-(2-methoxy-2-oxoethyl)cyclohexyl]phenyl}amino)(oxo)acetate

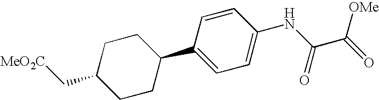

Methyl chloro(oxo)acetate (0.842 mL) was added to a stirred solution of methyl [trans-4-(4-aminophenyl)cyclohexyl]acetate (Intermediate 8; 1.74 g) and pyridine (0.689 mL) in DCM (50 mL) at 0° C. After the addition was complete the mixture was allowed to warm to ambient temperature and stirred for 64 hours. The solution was diluted with DCM (100 mL), washed with water (50 mL) and brine (50 mL), then dried and concentrated in vacuo to give the title compound as a solid (2.267 g): ¹H NMR: 7.60 (2H, d), 7.18 (2H, d), 3.83 (3H, s), 3.58 (3H, s), 2.58-35 (1H+DMSO, m), 2.21 (2H, d), 1.75 (5H, m), 1.43 (2H, m), 1.12 (2H, m); MS (M-H)⁻ 332.

### Intermediate 12: Methyl [trans-4-(4-{[hydrazino(oxo)acetyl]amino}phenyl)cyclohexyl]acetate

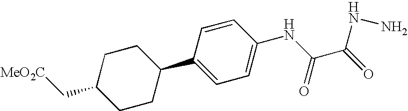

Hydrazine hydrate (0.361 mL) was added to a stirred solution of methyl ({4-[trans-4-(2-methoxy-2-oxoethyl)cyclohexyl]phenyl}amino)(oxo)acetate (Intermediate 9, 2260 mg) in EtOH (50 mL). The mixture was stirred for 1 hour. The precipitate was filtered off, washed with Et₂O, and dried under vacuum overnight to give the title compound as a solid (1.845 g):

¹H NMR: 10.44 (1H, s), 10.20 (1H, s), 7.70 (2H, d), 7.21 (2H, d), 4.60 (2H, s), 3.60 (3H, s), 2.42 (1H, m), 1.79 (5H, m), 1.45 (2H, m), 1.11 (2H, m); MS MH⁺ 334.

1. A compound of formula (I) 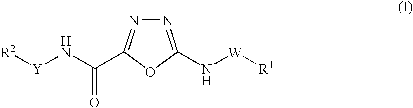
or a salt or prodrug thereof,
wherein
R¹ is an optionally substituted aryl or optionally substituted heteroaryl group, wherein the optional substituents are one or more groups selected from -Z^{a}, —X²—(CR³R⁴)_{q}-Z^{a}, —X²(CR³R⁴)ₐ—X³-Z^{a}, —(CR³R⁴)ₐX³-Z^{a} and R^{f};
W is selected from —C(O)—, —C(O)O—, —C(O)NH— and —C(O)(CR^{A}R^{B})ₖ—;
k is 0 to 4;
R^{A} and R^{B} are independently selected from hydrogen and (1-4C)alkyl, and/or two groups R^{A} and/or R^{B} are joined together to form a (3-8C)cycloalkyl ring;
Y is a direct bond or —(CR⁵R⁶)ₛ— or —X⁶(CR⁵R⁶)ₜ— wherein each R⁵ and R⁶ is independently selected from hydrogen, (1-4C)alkyl, hydroxy, halo, halo(1-4C)alkyl, amino, (1-4C)alkoxy, cyano(1-4C)alkoxy, (1-4C)haloalkoxy and (1-4C)alkylCONH—;
s is 1 to 6;
t is 1 to 6, provided that the X⁶ atom of the group —X⁶(CR⁵R⁶)ₜ— is attached to the R² group and that a single sp3 hybridised carbon atom does not carry two or more bonds to a heteroatom unless the heteroatom is a halo;
R² is an optionally substituted aryl, an optionally substituted (3-8C)cycloalkyl, an optionally substituted (5-12C)bicycloalkyl, an optionally substituted (6-12C)tricycloalkyl or an optionally substituted heterocyclic group, wherein the optional substituents are one or more groups selected from -Z, —X—(CR⁷R⁸)ᵤ-Z, —X—(CR⁷R⁸)ᵥ—X¹-Z, —(CR⁷R⁸)ᵥX¹-Z and R^{f};
Z and Z^{a} are independently selected from a hydrocarbyl group, heterocyclic group and a combination thereof, wherein Z and Z^{a} are optionally substituted on any available atom by one or more groups selected from R^{f} and —X⁷—(CR⁹R¹⁰)_{b}R¹¹;
X, X¹, X², X³, X⁶ and X⁷ are linking groups independently selected from —C(O)ₓ—, —O—, —S(O)_{y}—, —NR¹²—, —C(O)NR¹², —OC(O)NR¹²—, —CH═NO—, —NR¹²C(O)_{X}—, —NR¹²CONR¹³—, —S(O)₂NR¹²— and —NR¹²S(O)₂—;
x is 1 or 2;
y is 0, 1 or 2;
R¹² and R¹³ are independently selected from hydrogen and (1-6C)alkyl;
u and q are independently 0 to 6;
v, a and b are independently 1 to 6;
each R³, R⁴, R⁷, R⁸, R⁹ and R¹⁰ is independently selected from hydrogen, (1-4C)alkyl, hydroxy, halo, halo(1-4C)alkyl, amino, cyano(1-4C)alkoxy, (1-4C)haloalkoxy, (1-3C)alkylCONH—, carboxy, —SO₃H, —S(O)₂NHR¹³, —S(O)₂NHC(O)R¹³, —CH₂S(O)₂R¹³, —C(O)NHS(O)₂R¹³, —C(O)NHOH, —C(O)NHCN, —CF(CF₃)OH, —C(CF₃)₂OH, —P(O)(OH)₂, 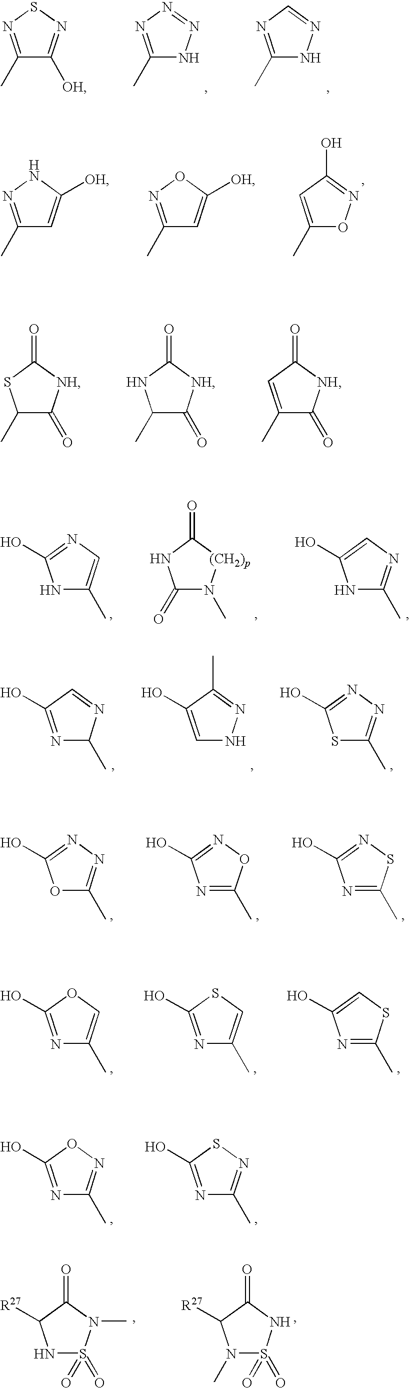 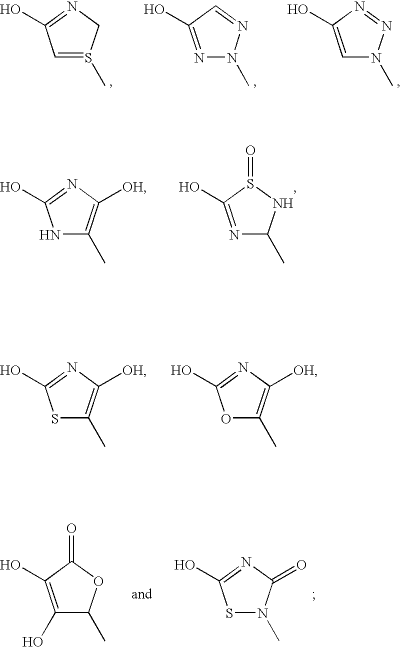
R¹³ is (1-6C)alkyl aryl or heteroaryl;
R²⁷ is hydrogen or (1-4C)alkyl;
R^{f} and R¹¹ are independently selected from halo, haloC₁₋₆alkyl, cyano, nitro, C(O)ₙR¹⁴, OR¹⁴, S(O)ₘR¹⁴, OS(O)₂R¹⁴, NR¹⁵R¹⁶, C(O)NR¹⁵R¹⁶, OC(O)NR¹⁵R¹⁶, —CH═NOR¹⁴, —NR¹⁵C(O)ₙR¹⁴, —NR¹⁴CONR¹⁵R¹⁶, —N═CR¹⁵R¹⁶, S(O)₂NR¹⁵R¹⁶, —NR¹⁵S(O)₂R¹⁶, —SO₃H, —S(O)₂NHR¹³, —S(O)₂NHC(O)R¹³, —CH₂S(O)₂R¹³, —C(O)NHS(O)₂R¹³, —C(O)NHOH, —C(O)NHCN, —CH(CF₃)OH, —C(CF₃)₂OH, —P(O)(OH)₂, 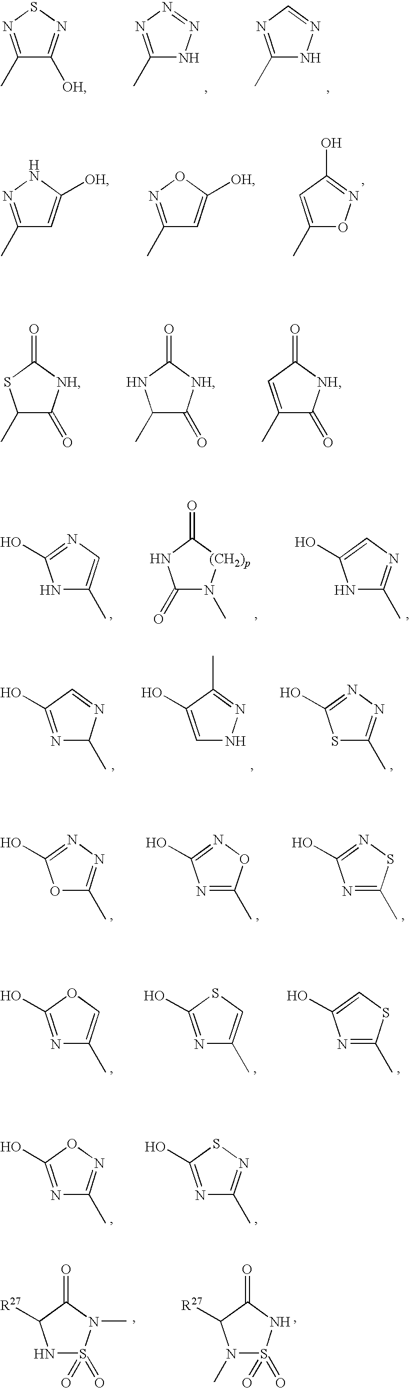 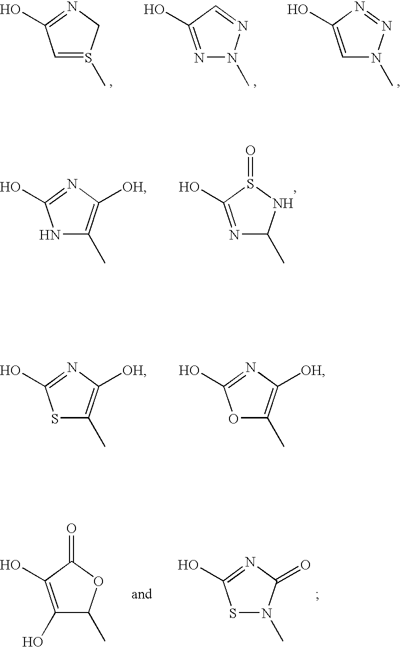
R¹³ is (1-6C)alkyl aryl or heteroaryl
R²⁷ is hydrogen or (1-4C)alkyl;
R¹⁴, R¹⁵ and R¹⁶ are independently hydrogen or optionally substituted hydrocarbyl or optionally substituted heterocyclyl, or R¹⁵ and R¹⁶ together with the nitrogen atom to which they are attached form an optionally substituted ring having from 3 to 10 atoms, which optionally contains further heteroatoms;
n is 1 or 2 and
m is 0, 1 or 2.

2. The compound of formula (I), or a salt or prodrug thereof, as claimed in claim 1, which is a compound of formula (IA): 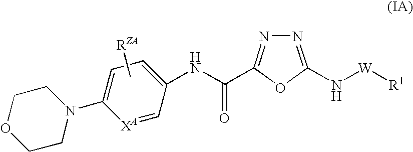
wherein
X^{A} is CH or N;
R^{ZA} is halo; and
W and R¹ are as defined in claim 1.

3. The compound of formula (I), or a salt or prodrug thereof, as claimed in claim 1, which is a compound of formula (IB): 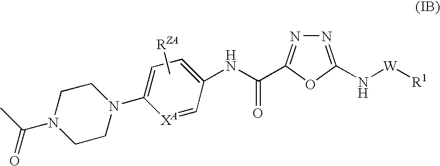
wherein
X^{A} is CH or N;
R^{ZA} is halo; and
W and R¹ are as defined in claim 1.

4. The compound of formula (I), or a salt or prodrug thereof, as claimed in claim 1, which is a compound of formula (IC): 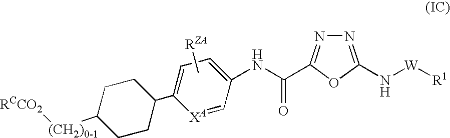
wherein
X^{A} is CH or N;
R^{ZA} is halo;
R^{C} is hydrogen or methyl; and
W and R¹ are as defined in claim 1.

5. The compound of formula (I), or a salt or prodrug thereof as claimed in claim 1, wherein W is selected from —C(O)—, —C(O)CH₂—, —C(O)CH(Me)—, —C(O)C(Me)₂- and —C(O)CR^{A}R^{B}—, wherein R^{A} and R^{B} together form a cyclopropyl, cyclobutyl, cyclopentyl or cyclohexyl ring.

6. The compound of formula (I), or a salt or prodrug thereof as claimed in claim 1, wherein R¹ is selected from an optionally substituted phenyl, naphthyl, thienyl, isoxazolyl, indolyl, benzothienyl, benzofuryl and quinoxalinyl group, wherein optional substances for R¹ include halo, (1-4C)alkyl, (1-4C)alkoxy, benzyloxy, cyano, nitro and halo(1-4C)alkoxy.

7. The compound of formula (I), or a salt or prodrug thereof as claimed in claim 1 which is any one or more of:
5-[(4-chlorobenzoyl)amino]-N-(3-fluoro-4-morpholin-4-ylphenyl)-1,3,4-oxadiazole-2-carboxamide;
5-{[2-(4-chlorophenyl)-2-methylpropanoyl]amino}-N-(4-morpholin-4-ylphenyl)-1,3,4-oxadiazole-2-carboxamide;
5-[(4-chlorobenzoyl)amino]-N-(6-morpholin-4-ylpyridin-3-yl)-1,3,4-oxadiazole-2-carboxamide;
methyl (trans-4-{4-[({5-[(4-chlorobenzoyl)amino]-1,3,4-oxadiazol-2-yl}carbonyl)amino]phenyl}cyclohexyl)acetate;
(trans-4-{4-[({5-[(4-chlorobenzoyl)amino]-1,3,4-oxadiazol-2-yl}carbonyl)amino]phenyl}cyclohexyl)acetic acid;
5-(benzoylamino)-N-(4-morpholin-4-ylphenyl)-1,3,4-oxadiazole-2-carboxamide;
5-[(3-fluorobenzoyl)amino]-N-(4-morpholin-4-ylphenyl)-1,3,4-oxadiazole-2-carboxamide;
5-[(3-methylbenzoyl)amino]-N-(4-morpholin-4-ylphenyl)-1,3,4-oxadiazole-2-carboxamide;
5-[(4-fluorobenzoyl)amino]-N-(4-morpholin-4-ylphenyl)-1,3,4-oxadiazole-2-carboxamide;
5-[(4-methoxybenzoyl)amino]-N-(4-morpholin-4-ylphenyl)-1,3,4-oxadiazole-2-carboxamide;
5-[(4-methylbenzoyl)amino]-N-(4-morpholin-4-ylphenyl)-1,3,4-oxadiazole-2-carboxamide;
5-[(4-cyanobenzoyl)amino]-N-(4-morpholin-4-ylphenyl)-1,3,4-oxadiazole-2-carboxamide;
N-(4-morpholin-4-ylphenyl)-5-(1-naphthoylamino)-1,3,4-oxadiazole-2-carboxamide;
N-(4-morpholin-4-ylphenyl)-5-(2-naphthoylamino)-1,3,4-oxadiazole-2-carboxamide;
N-(4-morpholin-4-ylphenyl)-5-[(4-nitrobenzoyl)amino]-1,3,4-oxadiazole-2-carboxamide;
N-(4-morpholin-4-ylphenyl)-5-[(phenylacetyl)amino]-1,3,4-oxadiazole-2-carboxamide;
N-(6-morpholin-4-ylpyridin-3-yl)-5-[(phenylacetyl)amino]-1,3,4-oxadiazole-2-carboxamide;
N-[4-(4-acetylpiperazin-1-yl)phenyl]-5-[(4-isopropoxybenzoyl)amino]-1,3,4-oxadiazole-2-carboxamide;
N-[4-(4-acetylpiperazin-1-yl)phenyl]-5-[(4-methylbenzoyl)amino]-1,3,4-oxadiazole-2-carboxamide;
N-[4-(4-acetylpiperazin-1-yl)phenyl]-5-[(4-chlorobenzoyl)amino]-1,3,4-oxadiazole-2-carboxamide;
N-[4-(4-acetylpiperazin-1-yl)phenyl]-5-[(4-tert-butylbenzoyl)amino]-1,3,4-oxadiazole-2-carboxamide;
N-[4-(4-acetylpiperazin-1-yl)phenyl]-5-[(4-methoxybenzoyl)amino]-1,3,4-oxadiazole-2-carboxamide;
N-[4-(4-acetylpiperazin-1-yl)phenyl]-5-{[4-(difluoromethoxy)benzoyl]amino}-1,3,4-oxadiazole-2-carboxamide;
5-{[2-(4-chlorophenyl)-2-methylpropanoyl]amino}-N-(4-morpholin-4-ylphenyl)-1,3,4-oxadiazole-2-carboxamide;
5-({[1-(2,4-dichlorophenyl)cyclopropyl]carbonyl}amino)-N-(4-morpholin-4-ylphenyl)-1,3,4-oxadiazole-2-carboxamide;
5-({[1-(4-chlorophenyl)cyclobutyl]carbonyl}amino)-N-(4-morpholin-4-ylphenyl)-1,3,4-oxadiazole-2-carboxamide;
5-({[1-(4-chlorophenyl)cyclopentyl]carbonyl}amino)-N-(4-morpholin-4-ylphenyl)-1,3,4-oxadiazole-2-carboxamide;
N-(4-morpholin-4-ylphenyl)-5-{[(1-phenylcyclopentyl)carbonyl]amino}-1,3,4-oxadiazole-2-carboxamide;
5-{[2-(4-chlorophenyl)-2-methylpropanoyl]amino}-N-(6-morpholin-4-ylpyridin-3-yl)-1,3,4-oxadiazole-2-carboxamide;
N-(6-morpholin-4-ylpyridin-3-yl)-5-{[(1-phenylcyclopentyl)carbonyl]amino}-1,3,4-oxadiazole-2-carboxamide;
5-({[1-(3-fluorophenyl)cyclopentyl]carbonyl}amino)-N-(6-morpholin-4-ylpyridin-3-yl)-1,3,4-oxadiazole-2-carboxamide;
5-({[1-(2-fluorophenyl)cyclopentyl]carbonyl}amino)-N-(6-morpholin-4-ylpyridin-3-yl)-1,3,4-oxadiazole-2-carboxamide;
N-[4-(4-acetylpiperazin-1-yl)phenyl]-5-[(4-benzyloxybenzoyl)amino]-1,3,4-oxadiazole-2-carboxamide;
N-[4-(4-acetylpiperazin-1-yl)phenyl]-5-[(3-isobutoxybenzoyl)amino]-1,3,4-oxadiazole-2-carboxamide;
N-[4-(4-acetylpiperazin-1-yl)phenyl]-5-[(3-isopropoxybenzoyl)amino]-1,3,4-oxadiazole-2-carboxamide;
N-[4-(4-acetylpiperazin-1-yl)phenyl]-5-[(2-ethylbenzoyl)amino]-1,3,4-oxadiazole-2-carboxamide;
N-[4-(4-acetylpiperazin-1-yl)phenyl]-5-{[3-(difluoromethoxy)benzoyl]amino}-1,3,4-oxadiazole-2-carboxamide;
N-[4-(4-acetylpiperazin-1-yl)phenyl]-5-({[1-(4-chlorophenyl)cyclohexyl]carbonyl}amino)-1,3,4-oxadiazole-2-carboxamide;
N-[4-(4-acetylpiperazin-1-yl)phenyl]-5-({[1-(4-chlorophenyl)cyclopentyl]carbonyl}amino)-1,3,4-oxadiazole-2-carboxamide;
N-[4-(4-acetylpiperazin-1-yl)phenyl]-5-({[1-(4-chlorophenyl)cyclobutyl]carbonyl}amino)-1,3,4-oxadiazole-2-carboxamide;
N-[4-(4-acetylpiperazin-1-yl)phenyl]-5-({[1-(3-fluorophenyl)cyclopentyl]carbonyl}amino)-1,3,4-oxadiazole-2-carboxamide;
N-[4-(4-acetylpiperazin-1-yl)phenyl]-5-({[1-(2-fluorophenyl)cyclopentyl]carbonyl}amino)-1,3,4-oxadiazole-2-carboxamide;
N-[4-(4-acetylpiperazin-1-yl)phenyl]-5-({[1-(4-fluorophenyl)cyclopentyl]carbonyl}amino)-1,3,4-oxadiazole-2-carboxamide:
N-[4-(4-acetylpiperazin-1-yl)phenyl]-5-{[(1-phenylcyclopentyl)carbonyl]amino}-1,3,4-oxadiazole-2-carboxamide;
N-[4-(4-acetylpiperazin-1-yl)phenyl]-5-{[2-(4-chlorophenyl)-2-methylpropanoyl]amino}-1,3,4-oxadiazole-2-carboxamide;
N-[4-(4-acetylpiperazin-1-yl)phenyl]-5-({[1-(4-methoxyphenyl)cyclopentyl]carbonyl}amino)-1,3,4-oxadiazole-2-carboxamide;
N-[4-(4-acetylpiperazin-1-yl)phenyl]-5-({[1-(4-chlorophenyl)cyclopropyl]carbonyl}amino)-1,3,4-oxadiazole-2-carboxamide;
N-[4-(4-acetylpiperazin-1-yl)phenyl]-5-{[(1-phenylcyclopropyl)carbonyl]amino}-1,3,4-oxadiazole-2-carboxamide;
N-[4-(4-acetylpiperazin-1-yl)phenyl]-5-{[(2S)-2-phenylpropanoyl]aminol}-1,3,4-oxadiazole-2-carboxamide;
N-[4-(4-acetylpiperazin-1-yl)phenyl]-5-({[1-(4-methoxyphenyl)cyclopropyl]carbonyl}amino)-1,3,4-oxadiazole-2-carboxamide;
N-[4-(4-acetylpiperazin-1-yl)phenyl]-5-[(2-thienylacetyl)amino]-1,3,4-oxadiazole-2-carboxamide;
N-[4-(4-acetylpiperazin-1-yl)phenyl]-5-[(3-thienylacetyl)amino]-1,3,4-oxadiazole-2-carboxamide;
N-[4-(4-acetylpiperazin-1-yl)phenyl]-5-{[(1-methyl-1H-indol-3-yl)acetyl]amino}-1,3,4-oxadiazole-2-carboxamide;
N-[4-(4-acetylpiperazin-1-yl)phenyl]-5-[(1-benzothien-3-ylacetyl)amino]-1,3,4-oxadiazole-2-carboxamide;
N-[4-(4-acetylpiperazin-1-yl)phenyl]-5-[(1-benzothien-2-ylcarbonyl)amino]-1,3,4-oxadiazole-2-carboxamide;
N-[4-(4-acetylpiperazin-1-yl)phenyl]-5-{[(5-methylisoxazol-3′-yl)carbonyl}amino]-1,3,4-oxadiazole-2-carboxamide;
N-[4-(4-acetylpiperazin-1-yl)phenyl]-5-[(2-thienylcarbonyl)amino]-1,3,4-oxadiazole-2-carboxamide;
N-[4-(4-acetylpiperazin-1-yl)phenyl]-5-{[(5-methyl-2-thienyl)carbonyl]amino}-1,3,4-oxadiazole-2-carboxamide;
N-[4-(4-acetylpiperazin-1-yl)phenyl]-5-[(1-benzofuran-2-ylcarbonyl)amino]-1,3,4-oxadiazole-2-carboxamide; and
N-[5-({[4-(4-acetylpiperazin-1-yl)phenyl]amino}carbonyl)-1,3,4-oxadiazol-2-yl]quinoxaline-2-carboxamide.

8. (canceled)

9. A method for treating diabetes mellitus and/or obesity in a warm-blooded animal comprising administering to an animal in need thereof a therapeutically effective amount of a compound as claimed in claim 1 or a pharmaceutically acceptable salt or prodrug thereof.

10. (canceled)

11. A method of inhibiting DGAT1 activity in a warm-blooded animal comprising administering to an animal in need thereof a therapeutically effective amount of a compound as claimed in claim 1, or a pharmaceutically-acceptable salt or prodrug thereof.

12. A pharmaceutical composition comprising compound as claimed in claim 1, or a pharmaceutically-acceptable salt thereof, in association with a pharmaceutically-acceptable excipient or carrier.

13. A process for preparing a compound as claimed in claim 1 comprising
a process a) to c) as follows, wherein all variables are as defined in claim 1 for a compound of formula (I) unless otherwise stated:
a) reacting a compound of formula (I) to form another compound of formula (I);
b) where Y is not a direct bond or R² is not aromatic, by reacting an amine of formula (2) with a carboxylate salt of formula (3); 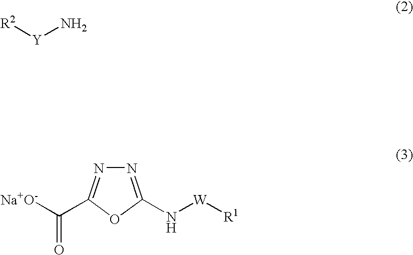
c) cyclising a compound of formula (4) wherein X is O or S and 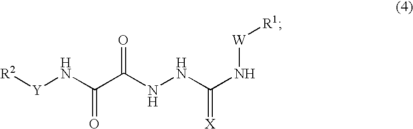
and optionally:
i) removing any protecting groups;
ii) forming a salt; and/or
iii) forming a prodrug thereof.
